(19) United States
(12) Patent Application Publication
(10) Pub. No.: US 2022/0089733 A1
(43) Pub. Date: Mar. 24, 2022

(54) NOVEL APPROACH FOR TREATMENT OF CANCER USING IMMUNOMODULATION

(71) Applicant: BioXcel Therapeutics, Inc., New Haven, CT (US)

(72) Inventor: Vincent J. O'NEILL, New Haven, CT (US)

(21) Appl. No.: 17/312,368

(22) PCT Filed: Dec. 10, 2019
(86) PCT No.: PCT/US2019/065465

§ 371 Date: Jun. 9, 2021

Related U.S. Application Data
(60) Provisional application No. 62/924,429, filed on Oct. 22, 2019.
(60) Provisional application No. 62/777,352, filed on Dec. 10, 2018.

(51) Int. Cl. C07K 16/28; A61P 35/00; A61K 31/69

(52) U.S. Cl. CPC C07K 16/2818; A61K 2039/505; A61K 31/69; A61P 35/00

(57) ABSTRACT

The present disclosure provides a regimen for treating a subject afflicted with prostate cancer by administering Talabostat or a pharmaceutically acceptable salt thereof and Pembrolizumab. The present disclosure is based on the discovery that the combination of Talabostat or a pharmaceutically acceptable salt thereof and Pembrolizumab in a specific treatment regimen is a very effective therapy to treat subjects afflicted with prostate cancer.

### CROSS REFERENCE TO RELATED APPLICATIONS

This application claims priority to U.S. Provisional Application Nos. 62/777,352, filed Dec. 10, 2018, and 62/924,429, filed Oct. 22, 2019, the contents of which are each incorporated in their entireties for all purposes.

### FIELD

The present disclosure is in the field of immune-oncology, and more specifically relates to a treatment regimen comprising Talabostat or a pharmaceutically acceptable salt thereof and Pembrolizumab to treat prostate cancer.

### BACKGROUND

Cancer is a multistep process that begins with minor pre-neoplastic changes, which may progress to neoplasia, the neoplastic lesions possibly developing an increasing capacity for invasion, growth, metastasis, and heterogeneity. Current therapies for the treatment of cancer involve surgery, hormonal therapy, radiation therapy, chemotherapy and immunotherapy. Immunotherapy for the treatment of cancer has evolved alongside our improved understanding of the immune system. In particular, an appreciation of the ability of cancer cells to subvert the antitumor immune response has provided a rationale for the development of novel immunotherapies that target immune checkpoints responsible for tumor cells escaping detection and destruction by the immune system.

Such immune escape mechanisms are mediated either directly by the tumor cells or by the tumor microenvironment. Tumor cells are known to express membrane proteins, secreted products, enzymes, anti-inflammatory cytokines, and chemokines to produce changes in their genome that aid in immune evasion and immune inhibition. At the same time, a key role is played by the tumor microenvironment.

Immune checkpoint molecules such as PD-1, PD-L1, CTLA-4 are cell surface signaling receptors that play important roles in modulating the T-cell response in the tumor microenvironment. Tumor cells have been shown to utilize these checkpoints to their benefit by up-regulating their expression and activity. Therefore, immune checkpoint inhibitors have been developed which can unleash the immune system's cancer-destroying properties. Recent discoveries have identified immune checkpoints or targets like PD-1, PD-L1, PD-L2, CTLA4, TIM3, LAG3, CCR4, OX40, OX40L, IDO, and A2AR as proteins responsible for immune evasion, acting as “brakes” of the immune system. Specific immune checkpoint inhibitors, including antibodies against CTLA-4, the PD-1 receptor and its ligand PD-L1 have produced impressive results in the clinic, leading to FDA approvals for Yervoy® (Ipilimumab; CTLA-4 antagonist), Opdivo® (Nivolumab; PD-1 antagonist) and Keytruda® (Pembrolizumab; PD-1 antagonist) in multiple tumor indications and with on-going clinical trials in many more.

Unfortunately, checkpoint inhibitors suffer from several limitations. Only a minority of patients treated with checkpoint inhibitors exhibit robust anti-tumor responses, and most responses are partial and temporary. Often patients initially respond, but then relapse due to the emergence of resistant pathways, which mainly occur due to the generation by the tumor cells of a non-immune permissive microenvironment.

The combination of the two checkpoint inhibitors, Ipilimumab and Nivolumab, have shown an increase in the response rate in melanoma patients from 11% and 32% seen with the respective monotherapy to 60% with the combination. Unfortunately, this combination has the significant drawback of high toxicity related to an excessive immune response, leading to pneumonitis, hepatitis, colitis and other immune related disorders.

Talabostat also known as PT-100 (Val-boroPro; L-valinyl-L-boroproline), was originally developed by Point Therapeutics, during 2000 to 2007. It is an orally available synthetic selective inhibitor of dipeptidyl peptidases like FAP and DPP8 and DPP9. The stereoisomer of the Talabostat molecule disclosed in the U.S. Pat. No. 6,825,169 while its oral formulation such as tablet, capsule, lozenges is disclosed in the U.S. Pat. No. 7,265,118.

Talabostat plays an important role in immune evasion and regulates both innate and/or acquired immunity. However, Talabostat has been reported to exhibit a number of side effects at therapeutically effective doses, with the most common adverse events being edema/peripheral swelling, hypotension, hypovolemia, and dizziness. These reported adverse events, as well as insufficient primary and secondary outcomes in certain cancer clinical trials, have led to the limited use of Talabostat as an anti-cancer agent.

In our U.S. Patent Application Publication No. 2017/0266280A1 (incorporated herein by reference in its entirety), we disclose the novel discovery that the combination of a selective dipeptidyl peptidase (DPP) inhibitor such as Talabostat with an immune checkpoint inhibitor is effective in treating cancer. Surprisingly, the combination is effective without concomitant treatment-limiting side effects. The present disclosure is based on the new discovery that Talabostat may be particularly effective in treating prostate cancer in combination with the PD-1 antagonist Pembrolizumab when both are administered at a particular treatment regimen.

Accordingly, the main object of the present disclosure is to provide improved therapies for treating prostate cancer using a novel treatment regimen comprising Talabostat or a pharmaceutically acceptable salt thereof and Pembrolizumab. Said treatment regimen may also be effective for treating one or more other solid tumors, such as advanced solid tumors for which Pembrolizumab has been demonstrated to have, or may be expected to have, a beneficial anti-cancer effect.

### SUMMARY

The present disclosure is based on the discovery that the combination of Talabostat or a pharmaceutically acceptable salt thereof and Pembrolizumab in a specific treatment regimen is a very effective therapy to treat subjects afflicted with prostate cancer. In particular, the dosage amount of each active used and the dosing schedule selected leads to a very effective treatment of prostate cancer (e.g. small cell neuroendocrine prostate cancer; SCNC).

Thus, in the principal aspect, the present disclosure provides a regimen for treating prostate cancer in a subject in need thereof, comprising administering to said subject, as separate pharmaceutical formulations, an effective amount of Talabostat or a pharmaceutically acceptable salt thereof and an effective amount of Pembrolizumab.

Another aspect provides a method of treating prostate cancer comprising administering to a subject in need thereof, as separate pharmaceutical formulations, an effective amount of Talabostat or a pharmaceutically acceptable salt thereof and an effective amount of Pembrolizumab.

In a particular aspect, the separate pharmaceutical formulations of Talabostat or a pharmaceutically acceptable salt thereof and Pembrolizumab are administered to the subject at relevant times, and in suitable amounts, during one or more treatment cycles of about 21 days, to maximize their combined immunotherapeutic effect.

A further aspect provides a method of enhancing an immune response in a subject suffering from prostate cancer, the method comprising administering to said subject a regimen comprising, as separate pharmaceutical formulations, an effective amount of Talabostat or a pharmaceutically acceptable salt thereof and an effective amount of Pembrolizumab.

In some aspects, the present disclosure provides a first pharmaceutical formulation comprising Talabostat or a pharmaceutically acceptable salt thereof for use in combination with a separate second pharmaceutical formulation of Pembrolizumab to treat prostate cancer, wherein said first pharmaceutical formulation comprises Talabostat or a pharmaceutically acceptable salt thereof together with one or more pharmaceutically acceptable carriers or adjuvants and said second pharmaceutical formulation comprises Pembrolizumab together with one or more pharmaceutically acceptable carriers or adjuvants.

In another aspect, the present disclosure provides a method of enhancing an innate immune response in a subject afflicted with prostate cancer, the method comprising administering to said subject, as separate pharmaceutical formulations, an effective amount of Talabostat or a pharmaceutically acceptable salt thereof and an effective amount of Pembrolizumab, wherein the enhanced innate immune response is associated with increased tumoricidal natural killer cells and macrophages, as well as the activity of NK cells and CD8+ T cells.

In a further aspect, the present disclosure provides a method of enhancing an innate immune response in a subject afflicted with prostate cancer, the method comprising administering to said subject, as separate pharmaceutical formulations, an effective amount of Talabostat or a pharmaceutically acceptable salt thereof and an effective amount of Pembrolizumab, wherein the enhanced innate immune response is associated with suppression of T-regulatory cells.

In some aspects, the present disclosure provides a kit for use in the treatment of prostate cancer, said kit comprising:

(i) a first pharmaceutical formulation comprising Talabostat or a pharmaceutically acceptable salt thereof;
(ii) a second pharmaceutical formulation comprising Pembrolizumab and
(iii) instructions for using said first and second pharmaceutical formulations according to the methods and regimen described herein

Other features, objects, and advantages of the disclosure will be apparent from the description and drawings, and from the claims.

### BRIEF DESCRIPTION OF THE DRAWINGS

FIG. 1: shows the scheme for administering Talabostat mesylate and Pembrolizumab to subjects with SCNC during the treatment Lead-in Stage and the Efficacy Stage.

FIG. 2: shows the prostate specific antigen (PSA) levels in three subjects in Cohort 1 during 4 or 5 treatment cycles.

FIG. 3: shows the scheme for administering Talabostat mesylate and Pembrolizumab to subjects with advanced solid cancers during the treatment Lead-in Stage and the Efficacy Stage.

### DETAILED DESCRIPTION

In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### ABBREVIATIONS

As used herein, the following abbreviations have the following meanings:

A2AR: A2A adenosine receptor
ADT: Androgen deprivation therapy
ALK: Anaplastic lymphoma kinase
ALT: Alanine aminotransferase
ANC: Absolute neutrophil count
AR: Androgen receptor
AST: Aspartate aminotransferase
AUC: Area under the plasma concentration-time curve
AUC₀₋ₗₐₛₜ: Area under the plasma concentration time curve for the last measurable concentration
BS: Bone scintigraphy
BUN: Blood urea nitrogen
CAF: Cancer associated fibroblast
CLL: Chronic lymphocytic leukemia
CR: Complete response
CRPC: Castration-resistant prostate cancer
CT: computed tomography
CTC: Circulating tumor cells
ctDNA: Circulating tumor DNA
CTLA4: Cytotoxic T-lymphocyte associated protein 4
CPS: Combined positive scores
DPP: Dipeptidyl peptidase
DKA: Diabetic ketoacidosis
DLT: Dose limiting toxicity
DOR: Duration of response

### DSRC: Data Safety Review Committee

EGFR: Epidermal growth factor receptor

### ECOG: Eastern Cooperative Oncology Group

eCRF: Electronic case report form

### EOT: End of Treatment

FAP: Fibroblast activation protein
GM-CSF: Granulocyte-macrophage colony-stimulating factor
G-CSF: Granulocyte-colony stimulating factor

### OCP: Good Clinical Practice

HER2: Human epidermal growth factor receptor 2
HCC: hepatocellular carcinoma
ICI: Immune check point inhibitor
IC50: Half maximal inhibitory concentration

### ICH: International Council for Harmonisation

### IEC: Independent Ethics Committee

### IL: Interleukin

IDO: Indoleamine 2,3-dioxygenase
IMT: Inflammatory myofibroblastic tumor
IrCR: Immune-related complete disease
irPR: Immune-related partial response
irSD: Immune-related stable disease

### IND: Investigational New Drug (Application)

### IRB: Institutional Review Board

iRECIST: Immune Response Evaluation Criteria In Solid tumors

### ITT: Intent-to-Treat

LAG3: Lymphocyte activation gene 3 protein
LDH: Lactate dehydrogenase
LHRH: Luteinizing hormone-releasing hormone
MSI-H: Microsatellite instability-high
MDSC: Myeloid derived suppressor cell

### MedDRA: Medical Dictionary for Regulatory Activities

MRI: Magnetic resonance imaging
mRNA: Messenger ribonucleic acid
NK: Natural killer

### NCI CTCAE: National Cancer Institute Common Terminology Criteria for Adverse Events

NEPC: Neuroendocrine prostate cancer
NHL: Non-Hodgkin's lymphoma
NSCLC: Non-small cell lung cancer OS: Overall survival

### PCWG3: Prostate Cancer Working Group 3

PD: Progressive disease
PD-1: Programmed cell death 1
PD L1: Programmed death ligand 1
PD L2: Programmed death ligand 2
PFS: Progression-free survival
PR: Partial response

### PD-1: Programmed Cell Death 1

### Q.D: Quaque die

QTcB: QT interval corrected for heart rate using Bazett's formula

### RECIST: Response Evaluation Criteria In Solid Tumors

rPFS: Radiographic progression-free survival
SD: Stable disease
SAE: Serious adverse event

### SAP: Statistical Analysis Plan

SJS: Stevens-Johnson syndrome
SCNC: Small cell neuroendocrine prostate cancer
sHASEGP: Soluble neutral-active hyaluronidase glycoproteins
TIM3: T-cell immunoglobulin and mucin-domain containing-3
Treg: Regulatory T cells or T-regulatory cells

### TPS: Tumor Proportion Score

TRAE: Treatment related adverse events
TEN: Toxic epidermal necrolysis
T1DM: Type 1 diabetes mellitus
Tmax: Time of maximum observed concentration
ULN: Upper limit of normal

The therapeutic agents Talabostat and Pembrolizumab as intended for use in the present disclosure are described below:

### 1. THERAPEUTIC AGENTS

### a) Talabostat or a Pharmaceutically Acceptable Salt Thereof:

Talabostat is referred to interchangeably as PT-100, Talabostat (USAN), and [(2R)—I—I [(2S)-2-amino-3-methyl-1-oxobutyl]-2-pyrrolidinyl] boronic acid. Talabostat has a CAS registration number of 149682-77-9. Talabostat, also known as Val-boro-pro (L-valinyl-L-boroproline), is disclosed in PCT Appl. Publication No. 1989/003223. The IUPAC name of talabostat is [(2R)-1-[(2S)-2-amino-3-methylbutanoyl]pyrrolidin-2-yl]boronic acid. Talabostat (PubChem ID: 6918572), or a pharmaceutically acceptable salt thereof, such as, for example, talabostat mesylate (PubChem CID: 1152248). In some aspects, the free base may be used. In other aspects, the Talabostat or a pharmaceutically acceptable salt thereof may be a solvate. In most clinical formulations, Talabostat is provided as a salt form, e.g. Talabostat mesylate. Talabostat has two chiral centers with a R, S configuration. Talabostat or a pharmaceutically acceptable salt thereof can exist as both linear and cyclic forms (RI Snow et al., J. Am. Chem. Soc., 1994, 116 (24), pp 10860-10869).

Talabostat or a pharmaceutically acceptable salt thereof is effective for the treatment of cancer by modulating multiple intracellular and extracellular dipeptidyl peptidases. More specifically, intracellular and extracellular dipeptidyl peptidases comprise Fibroblast Activation Protein, DPP 8/9, CD26/DPP4 and DPP2. Talabostat or a pharmaceutically acceptable salt thereof has a dual mechanism of action which includes stromal targeted activity via FAP inhibition and targeted immunostimulatory activity via DPP 8/9 inhibition. Talabostat inhibits FAP enzymatic activity thereby suppressing tumor growth. Talabostat or a pharmaceutically acceptable salt thereof also inhibits DPP8/9 thereby inducing an IL 10 response (via caspase-1) in the stroma of tumor and lymph nodes. Talabostat's dual mechanism of action introduces a novel approach to the treatment of cancer because it combines both tumor-targeted and immune-stimulatory activity in a single agent.

### b) Pembrolizumab:

Pembrolizumab (also known as MK-3475, Lambrolizumab, Keytruda®, and SCH-900475) is a humanized antibody, which targets the PD-1 receptor of lymphocytes, thereby blocking PD-1 inhibitory signal transduction. Pembrolizumab may be readily procured from the marketplace.

### 2. METHODS OF USE

The present disclosure is based, in part, on an improved regimen to treat prostate cancer (e.g. SCNC) using effective amounts of Talabostat or a pharmaceutically acceptable salt thereof and Pembrolizumab. The combination of Talabostat or a pharmaceutically acceptable salt thereof and Pembrolizumab as used herein may produce an overall enhanced anti-cancer effect, such as improved T-cell priming, increased T cell stimulation, increased infiltration of neutrophil and macrophages across tumor microenvironment, decreased tumor volume, increased activation of natural killer cells, enhanced activation of dendritic cells, synergistic increase in pro-inflammatory cytokine (IL′. IL2, IL18, IFN gamma, IL6, IL12p40, IL 15, IL 7, G-CSF and GM-CSF), enhanced anti-tumor memory response, reduced metastasis and reduced toxicity.

In one embodiment, the present disclosure provides a combination comprising an effective amount of Talabostat or a pharmaceutically acceptable salt thereof and an effective amount of Pembrolizumab administered to a subject afflicted with prostate cancer (e.g. SCNC) in one or more treatment cycles, each cycle of about 21 days duration.

An advantage of using the particular regimen of Talabostat or a pharmaceutically acceptable salt thereof and Pembrolizumab of this disclosure is in the curtailment of the progression of prostate cancer (e.g. SCNC), reduction in tumor burden, reduced metastasis and/or inducement of tumor regression in a subject.

Thus, in one aspect, the present disclosure relates to a combination of Talabostat or a pharmaceutically acceptable salt thereof and Pembrolizumab administered to a subject afflicted with prostate cancer (e.g. SCNC) in a particular treatment regimen to promote an effective anti-tumor response.

In one embodiment, provided herein is a method of delaying progression of or preventing or delaying tumor recurrence, tumor growth or spread of tumor, in a subject afflicted with prostate cancer (e.g. SCNC), the method comprising administering to the subject, as separate pharmaceutical formulations, effective amounts of Talabostat or a pharmaceutically acceptable salt thereof and Pembrolizumab over one or more treatment cycles, each cycle of about 21 days duration.

In another embodiment, provided herein is a method of enhancing immune function in a subject afflicted with prostate cancer (e.g. SCNC), the method comprising administering to the subject, as separate pharmaceutical formulations, effective amounts of Talabostat or a pharmaceutically acceptable salt thereof and Pembrolizumab over one or more treatment cycles, each cycle of about 21 days duration.

In a further embodiment, provided herein is a method for initiating, sustaining or enhancing an anti-tumor immune response in a subject afflicted with prostate cancer (e.g. SCNC), the method comprising administering to the subject, as separate pharmaceutical formulations, effective amounts of Talabostat or a pharmaceutically acceptable salt thereof and Pembrolizumab over one or more treatment cycles, each cycle of about 21 days duration.

In some embodiments, the treatment regimen described herein results in a sustained response in the subject after cessation of the treatment.

In some embodiments, the subject has a prostate cancer that may be at an early stage or a late stage. In a particular embodiment, the cancer is advanced malignant solid neoplasm. In another particular embodiment, the cancer is recurrent malignant solid neoplasm.

In some embodiments, the prostate cancer is metastatic.

In some embodiments, the subject is a human.

In another embodiment, the present disclosure provides a method for reducing the toxicity of Talabostat or a pharmaceutically acceptable salt thereof with a lower effective dose of Talabostat, the method comprising administering to a subject afflicted with prostate cancer (e.g. SCNC), as separate pharmaceutical formulations, Talabostat or a pharmaceutically acceptable salt thereof and Pembrolizumab in the treatment regimen described herein.

In a further embodiment, the treatment regimen of the present disclosure comprises the use, as separate pharmaceutical formulations, of effective amounts of Talabostat or a pharmaceutically acceptable salt thereof and Pembrolizumab to produce an increased innate immune response as compared to the innate immune response when the subject is administered Talabostat alone. The innate immune response may be increased by infiltration of innate immune cells, in particular macrophages into the blood, and NK-cells into the tumor. Further, the present treatment regimen comprising effective amounts of Talabostat or a pharmaceutically acceptable salt thereof and Pembrolizumab can produce suppression of the Treg function that is greater than that obtained using Talabostat alone.

The present treatment regimen comprising effective amounts of Talabostat or a pharmaceutically acceptable salt thereof and Pembrolizumab can also significantly increase the tumor infiltration of immune sub-populations, such as NK-cells and macrophages, compared to monotherapies using Talabostat or a pharmaceutically acceptable salt thereof and Pembrolizumab.

### 3. TREATMENT REGIMENS

In some embodiments, during each treatment cycle of about 21 days, Talabostat or a pharmaceutically acceptable salt thereof may be administered at an effective dose on each of days 1 to 14 and Pembrolizumab may be administered at an effective dose on day 1. Said regimen is effective to treat a subject afflicted with prostate cancer (e.g. SCNC). The regimen herein disclosed for treating prostate cancer (e.g. SCNC) may also be used more generally to treat a subject with a solid tumor, e.g. an advanced solid tumor.

Tabalostat or a pharmaceutically acceptable salt thereof may be administered as a single daily dose in the regimen of this disclosure or, more particularly, as multiple dosage units to achieve an effective total daily dose to treat a subject afflicted with prostate cancer (e.g. SCNC). In alternate embodiment, Tabalostat or a pharmaceutically acceptable salt thereof may be administered twice a day in the dosage regimen of this disclosure to achieve an effective total daily dose to treat a subject afflicted with prostate cancer (e.g. SCNC).

Pembrolizumab may conveniently be administered as a single dose in the regimen of this disclosure to effectively treat a subject afflicted with prostate cancer (e.g. SCNC).

In certain embodiments, Pembrolizumab may be administered at a total dose of about 1 mg/kg to about 10 mg/kg per day, conveniently by injection (e.g., intravenously), most preferably as continuous infusion for 30 minutes. A suitable dose of Pembrolizumab administered intravenously in the treatment regimen of the present disclosure may conveniently be from about 100 mg to about 500 mg per day, e.g. about 200 mg per day.

In some embodiments, Pembrolizumab (MK-3475) is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of about 30 minutes.

In certain embodiments, Tabalostat or a pharmaceutically acceptable salt thereof may be administered at a total daily dose of about 0.001 mg/kg to about 0.1 mg/kg (e.g. about 0.001 mg/kg to about 0.035 mg/kg, or about 0.001 mg/kg to about 0.014 mg/kg), conveniently by the oral route (e.g. tablet). A suitable daily dose of Tabalostat or a pharmaceutically acceptable salt thereof administered orally via one or more (e.g. two or three) tablets in the treatment regimen of the present disclosure may conveniently be from about 0.1 mg to about 1 mg (e.g. about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, preferably about 0.1 mg to about 0.6 mg, more preferably about 0.4 mg to about 0.6 mg). In a particular embodiment, Talabostat or a pharmaceutically acceptable salt thereof may be administered orally at a dose of about 0.3 mg twice daily in divided doses. In one particular embodiment, Talabostat or a pharmaceutically acceptable salt thereof may be administered orally at a dose of about 0.2 mg thrice daily in divided doses. In another particular embodiment, Talabostat or a pharmaceutically acceptable salt thereof may be administered orally twice daily, such as at a dose of about 0.4 mg in the morning and about 0.2 mg in the evening in a day.

Suitable treatment regimens for treating a human patient afflicted with prostate cancer (e.g. SCNC) include, for example, administering to the patient, as separate pharmaceutical formulations, an effective amount of each of Talabostat or a pharmaceutically acceptable salt thereof and Pembrolizumab, wherein the regimen comprises at least one administration cycle (e.g. 1, 2, 3, 4, 5, 6 or more cycles), wherein each cycle is a period of about 21 days, and wherein for each cycle, Talabostat or a pharmaceutically acceptable salt thereof is administered orally (e.g. by tablet) on each of days 1 to 14 and Pembrolizumab is administered intravenously on day 1.

In a particular embodiment, during one or more treatment cycles of about 21 days, Talabostat is administered on days 1 to 14 at a total daily dose of about 0.4 mg to about 0.6 mg and Pembrolizumab is administered on day 1 at a total dose of about 100 mg to about 500 mg per day, e.g. about 200 mg per day.

In another embodiment, the daily dose of Talabostat or a pharmaceutically acceptable salt thereof may be varied over time. For example, during the first cycle (including the Lead-in Stage) Talabostat or a pharmaceutically acceptable salt thereof may be administered at a lower daily dose than during subsequent cycles (e.g. Efficacy Stage). For example, Talabostat or a pharmaceutically acceptable salt thereof may conveniently be administered during the Lead-in Stage a daily dose of about 0.4 mg, and, if there are no side effects or other criteria preventing further treatment, the subject is allowed to enter the Efficacy Stage and administered, during Efficacy Stage, a daily dose of about 0.6 mg. In another embodiment, Talabostat or a pharmaceutically acceptable salt thereof may be administered during the Lead-in Stage a daily dose of about 0.6 mg and during the Efficacy Stage a daily dose of about 0.4 mg. In another embodiment, the patient is treated directly in the Efficacy Stage using a daily dose of about 0.4 mg or about 0.6 mg.

In other embodiments, Talabostat or a pharmaceutically acceptable salt thereof and Pembrolizumab are administered to a subject afflicted with prostate cancer (e.g. SCNC) in any desired number of treatment cycles as long as a clinical benefit is observed or until there is a complete response, confirmed progressive disease or unmanageable toxicity.

In further embodiments, the daily dosages of Talabostat or a pharmaceutically acceptable salt thereof and Pembrolizumab used according to the treatment regimen of this disclosure may be lower than the daily dosages of one or both of Talabostat or a pharmaceutically acceptable salt thereof and Pembrolizumab administered as single agents to treat a subject afflicted with prostate cancer (e.g. SCNC).

In some embodiments, the combination therapy is preferably administered for at least 12 weeks (three 4-week cycles or four 3-week cycles), more preferably at least 24 weeks, and even more preferably at least 2 to 4 weeks after the patient achieves a complete response.

In some embodiments, a single administration cycle comprises 21 days (21-day cycle). In specific embodiment, Talabostat mesylate is administered once daily (QD) on Days 1 to 14 of a 21-day cycle plus pembrolizumab 200 mg administered intravenously (IV) on Day 1 every 21 days.

In some embodiments, a single administration cycle comprises 21 days (21-day cycle). In specific embodiment, Talabostat mesylate is administered twice daily at a dose of 0.3 mg on Days 1 to 14 of a 21-day cycle plus pembrolizumab 200 mg administered intravenously (IV) on Day 1 every 21 days. In another embodiment, Talabostat mesylate is administered thrice daily at a dose of about 0.2 mg on Days 1 to 14 of a 21-day cycle plus pembrolizumab 200 mg administered intravenously (IV) on Day 1 every 21 days. In another embodiment, Talabostat mesylate is administered at a dose of about 0.4 mg in the morning and about 0.2 mg in the evening on Days 1 to 14 of a 21-day cycle plus pembrolizumab 200 mg administered intravenously (IV) on Day 1 every 21 days. In another embodiment, Talabostat mesylate is administered at a dose of about 0.2 mg in the morning and about 0.4 mg in the evening on Days 1 to 14 of a 21-day cycle plus pembrolizumab 200 mg administered intravenously (IV) on Day 1 every 21 days.

In some embodiments, a combination therapy of the disclosure is administered to a patient who has not been previously treated with a biotherapeutic or chemotherapeutic agent, i.e., is treatment-naive. In other embodiments, the combination therapy is administered to a patient who failed to achieve a sustained response after prior therapy with a biotherapeutic or chemotherapeutic agent, i.e., is treatment-experienced.

In some embodiments of any of the methods or regimen described herein, before the period of time, the subject was treated with Talabostat mesylate as a monotherapy, and, optionally, the prior treatment with the Talabostat mesylate as a monotherapy was unsuccessful. In some embodiments of any of the methods or regimen described herein, before the period of time, the patient was treated with Pembrolizumab, or a biosimilar thereof as a monotherapy, and optionally, the prior treatment with Pembrolizumab, or a biosimilar thereof was unsuccessful.

A suitable period of time can be determined by one skilled in the art (e.g., a physician). As can be appreciated in the art, a suitable period of time can be determined by one skilled in the of based on one or more of: the stage of disease in the patient, the mass and sex of the patient, clinical trial guidelines (e.g., those on the fda.gov website), and information on the approved drug label. For example a suitable period of time can be, e.g., from 1 week to 2 years, 1 week to 22 months, 1 week to 20 months, 1 week to 18 months, 1 week to 16 months, 1 week to 14 months, 1 week to 12 months, 1 week to 10 months, 1 week to 8 months, 1 week to 6 months, 1 week to 4 months 1 week to 2 months, 1 week to 1 month, 2 weeks to 2 years, 2 weeks to 22 months, 2 weeks to 20 months, 2 weeks to 18 months, 2 weeks to 16 months, 2 weeks to 14 months, 2 weeks to 12 months, 2 weeks to 10 months, 2 weeks to 8 months, 2 weeks to 6 months, 2 weeks to 4 months, 2 weeks to 2 months, 2 weeks to 1 month, 1 month to 2 years, 1 month to 22 months, 1 month to 20 months, 1 month to 18 months, 1 month to 16 months, 1 month to 14 months, 1 month to 12 months, 1 month to 10 months, 1 month to 8 months, 1 month to 6 months, 1 month to 4 months, 1 month to 2 months, 2 months to 2 years, 2 months to 22 months, 2 months to 20 months, 2 months to 18 months, 2 months to 16 months, 2 months to 14 months, 2 months to 12 months, 2 months to 10 months, 2 months to 8 months, 2 months to 6 months, 2 months to 4 months, 3 months to 2 years, 3 months to 22 months, 3 months to 20 months, 3 months to 18 months, 3 months to 16 months, 3 months to 14 months. 3 months to 12 months, 3 months to 10 months, 3 months to 8 months, 3 months to 6 months, 4 months to 2 years, 4 months to 22 months, 4 months to 20 months, 4 months to 18 months, 4 months to 16 months, 4 months to 14 months, 4 months to 12 months, 4 months to 10 months, 4 months to 8 months, 4 months to 6 months, 6 months to 2 years, 6 months to 22 months, 6 months to 20 months, 6 months to 18 months, 6 months to 16 months, 6 months to 14 months, 6 months to 12 months, 6 months to 10 months, 6 months to 8 months, 8 months to 2 years, 8 months to 22 months, 8 months to 20 months, 8 months to 18 months, 8 months to 16 months, 8 months to 14 months, 8 months to 12 months, 8 months to 10 months, 10 months to 2 years, 10 months to 22 months, 10 months to 20 months, 10 months to 18 months, 10 months to 16 months, 10 months to 14 months, 10 months to 12 months, 12 months to 2 years, 12 months to 22 months, 12 months to 20 months, 12 months to 18 months, 12 months to 16 months, or 12 months to 14 months, inclusive.

### 4. PHARMACEUTICAL FORMULATIONS

In one embodiment, the present disclosure provides for use in the treatment regimen herein a pharmaceutical formulation comprising an effective amount of Talabostat or a pharmaceutically acceptable salt thereof and an effective amount of Pembrolizumab together with one or more pharmaceutically acceptable carriers or adjuvants. Any of the pharmaceutically acceptable carriers or adjuvants described herein, or known in the art, may be used. As used herein, the term “pharmaceutical formulation” refers to a formulation comprising Talabostat or a pharmaceutically acceptable salt thereof or a formulation comprising Pembrolizumab, wherein each formulation also comprises one or more pharmaceutically acceptable carriers or adjuvants. Pharmaceutically acceptable carriers or adjuvants are well-known to those skilled in the art, and usually depend on the chosen route of administration.

In some embodiments, a first formulation comprising Talabostat or a pharmaceutically acceptable salt thereof and one or more pharmaceutically acceptable carriers or adjuvants and a second formulation comprising Pembrolizumab and one or more pharmaceutically acceptable carriers or adjuvants are administered according to the treatment regimen disclosed herein to produce a synergistic effect in treating a subject afflicted with prostate cancer (e.g. SCNC).

The pharmaceutical formulations may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions may be formulated as the injectable or infusible solutions. The formulation is in a form suitable for oral, intravenous, intraarterial, intramuscular, subcutaneous, parenteral, transmucosal, transdermal, or topical administration. The formulation may be formulated as an immediate, controlled, extended or delayed release composition.

In some embodiments, the formulation comprising Talabostat or a pharmaceutically acceptable salt thereof may be administered orally. In some embodiments, the formulation comprising Pembrolizumab may be administered parenterally. As used herein, the term “parenteral” includes subcutaneous, intravenous, intra-arterial, intraperitoneal, intracardiac, intrathecal, and intramuscular injection, as well as infusion injections.

Liquid pharmaceutical formulations for parenteral administration may be formulated for administration by injection or continuous infusion. In some embodiments, parenteral formulations can include prefilled syringes, vials, powder for infusion for reconstitution, concentrate for infusion to be diluted before delivery (ready to dilute), solutions (ready to use).

Injectable pharmaceutical formulations can be aqueous isotonic solutions or suspensions, and suppositories can be prepared from fatty emulsions or suspensions.

Pharmaceutical formulations formulated for parenteral administration (e.g. via intravenous injection) may conveniently include a liquid carrier, or may be reconstituted into a liquid solution or suspension for parenteral administration.

In general, such formulations typically comprise a pharmaceutically acceptable carrier or adjuvant. As used herein, the term “pharmaceutically acceptable” means approved by a government regulatory agency or listed in the U.S. Pharmacopoeia or another generally recognized pharmacopoeia for use in animals, particularly in humans.

Pharmaceutical formulations of Pembrolizumab for intravenous administration may be purchased from the marketplace or prepared using conventional formulation techniques. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride, hexamethonium chloride, benzalkonium chloride, benzethonium chloride, phenol, butyl or benzyl alcohol, chlorobutanol, thimerosal, alkyl parabens such as methyl or propyl paraben, catechol, resorcinol, cyclohexanol, 3-pentanol and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; chelating agents such as EDTA; monosaccharides, disaccharides, and other carbohydrates including sugars such as sucrose, mannitol, trehalose or sorbitol, glucose, mannose, or dextrins; salt-forming counter-ions such as sodium; metal complexes (for example, Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). The carrier can be a solvent or reconstitution medium or dispersion medium containing, for example, water, ethanol, polyol (for example, glycerol, propylene glycol, and liquid polyethylene glycol, and the like), and suitable mixtures thereof. More particularly, the pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, liquid polyethylene glycol, or the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the formulation includes isotonic agents, for example, sugars, polyalcohols, such as mannitol, sorbitol, or sodium chloride. For intravenous administration, suitable carriers include physiological saline, bacteriostatic water, Cremophor EL (BASF, Parsippany, N.J.) or phosphate buffered saline (PBS). Prolonged absorption of the injectable compositions can be brought about by including in the composition an agent which delays absorption, for example, aluminium monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Such articles of manufacture will preferably have labels or package inserts indicating that the associated compositions are useful for treating a subject suffering from or predisposed to autoimmune or neoplastic disorders. The pharmaceutical formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

Solution or suspension formulations used for subcutaneous application typically include one or more of the following components: a sterile carrier such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical formulations of Talabostat or a pharmaceutically acceptable salt thereof for oral use herein may be administered, for example, in the form of tablets, capsules, powders, dispersible granules, sachets etc., or as aqueous solutions or suspensions, preferably tablets. Oral compositions generally include an inert carrier (for example, diluent) or an edible carrier. The formulations may be enclosed in a gelatin capsule or compressed into a tablet. Pharmaceutically compatible binding agents, and/or adjuvant materials can be included as part of the formulation. The tablets, pills, capsules, granules, sachets, troches and the like can contain any of the following ingredients, or compounds of a similar nature; a binder such as microcrystalline cellulose, gum tragacanth or gelatin; an excipient such as starch or lactose, a disintegrating agent such as alginic acid, primogel, or corn starch; a lubricant such as magnesium stearate or stearates; a glidant such as colloidal silicon dioxide; a sweetening agent such as sucrose or saccharin; or a flavouring agent such as peppermint, methyl salicylate, or orange flavouring.

In some embodiments, an oral pharmaceutical formulation comprising Talabostata pharmaceutically acceptable salt thereof described herein may comprise one or more pharmaceutically acceptable carriers or adjuvants selected from the group comprising a bulking agent, buffer, surfactant, and pH modifier. The pharmaceutical formulation may be adjusted to give an appropriate pH.

In a particular embodiment, Talabostat or a pharmaceutically acceptable salt thereof is formulated as a tablet for oral administration according to the treatment regimen of this disclosure. The pharmaceutical tablet may be an immediate release or a modified release tablet. Tablet may be in the form of matrix or coated form.

In certain embodiments, the various processes of making above mentioned formulations or compositions are included and such formulations can be manufactured by any of the processes known in the art.

An exemplary immediate release tablet comprises an effective amount of Talabostat or a pharmaceutically acceptable salt thereof and one or more pharmaceutically acceptable carriers selected from diluents, binders, disintegrants, glidants, lubricants, pH modifying agents and combinations thereof.

Diluents: one or more diluents comprise, but are not limited to dibasic calcium phosphate, pullulan, maltodextrin, isomalt, sugar pellets, mannitol, spray-dried mannitol, microcrystalline cellulose, dibasic calcium phosphate dihydrate, lactose, sugars, sorbitol, mixture of microcrystalline cellulose and guar gum (Avicel CE-15), mixture of mannitol, polyplasdone and syloid (Pharmaburst), mixture of mannitol, crospovidone and polyvinyl acetate (Ludiflash), isomalt, Panexcea, F-Melt, sucrose, calcium salts and similar inorganic salts, heavy magnesium carbonate and the like, and the mixtures thereof. Preferably, it is lactose or microcrystalline cellulose.

Binders: one or more binders comprise, but are not limited to, low-substituted hydroxypropyl cellulose, xanthan gum, polyvinylpyrrolidone (povidone), gelatin, sugars, glucose, natural gums, gums, synthetic celluloses, polymethacrylate, hydroxypropyl methylcellulose, hydroxypropyl cellulose, carboxymethyl cellulose, methyl cellulose, and other cellulose derivatives and the like, and the mixtures thereof. Preferably, the binder is polyvinylpyrrolidone or hydroxypropyl cellulose or hydroxypropyl methylcellulose.

Disintegrants: one or more binders comprise, but are not limited to, at least one or a mixture of sodium starch glycolate, croscarmellose sodium, crospovidone, sodium alginate, gums, starch, and magnesium aluminium silicate. Preferably, the disintegrant is sodium starch glycolate.

Lubricants: one or lubricants comprise, but are not limited to sodium stearyl fumarate, sodium lauryl sulphate, magnesium stearate, polyethylene glycol, metal stearates, hydrogenated castor oil and the like, and the mixtures thereof. Preferably, the lubricant is magnesium stearate.

Glidant: one or glidants comprise, but are not limited to, stearic acid, colloidal silicon dioxide, talc, aluminium silicate and the like, and the mixtures thereof. Preferably, it is talc.

pH modifying agents: one or more pH modifying agents comprise, but are not limited to organic acid or its salts like phosphoric acid, citric acid and the like.

In one embodiment, the relative percentages of the ingredients in tablet formulations of Talabostat is given below in Table 1:

TABLE 1

| Formulation Content | Amount (w/w %) |
|---|---|
| Talabostat as an API (available as | 0.01-2 |
| Talabostat mesylate) | |
| Binder | 1-50 |
| Disintegrant | 1-15 |
| Lubricant | 0.1-5 |
| Diluent | 30-98 |
| pH modifying agent | 0-15 |

An exemplary immediate release tablet of Talabostat mesylate is given below in Table 2:

TABLE 2

| | Preferred ranges | Amount |
|---|---|---|
| Formulation content | (w/w %) | (w/w %) |
| Talabostat mesylate (69% free | 0.01-2 | 0.145 |
| base) | | |
| Polyvinylpyrrolidone or | 1-50 | 1.00 |
| hydroxypropylcellulose or | | |
| hydroxypropylmethylcellulose | | |
| or pregelatinized starch as a | | |
| binder | | |
| Sodium starch glycolate or | 1-15 | 2.5 |
| crospovidone as a disintegrant | | |
| Stearic acid as a lubricant | 0.1-5 | 1.500 |
| Lactose as a diluent | 30-90 | 85.315 |
| Microcrystalline cellulose as a | 5-20 | 9.480 |
| diluent | | |
| Sodium phosphate monobasic, | 0-15 | 0.060 |
| monohydrate as a pH | | |
| modifying agent | | |
| Phosphoric acid as a pH | For pH adjustment | For pH adjustment |
| modifying agent | | |

In some embodiments, Talabostat or a pharmaceutically acceptable salt thereof may be formulated as a modified release matrix tablet. An exemplary extended release tablet comprises an effective amount of Talabostat or a pharmaceutically acceptable salt thereof and pharmaceutically-acceptable carrier or adjuvant are selected from the diluents, binders, modified release material, glidants, lubricants, colorants and combinations thereof. Alternatively, a modified release tablet comprises immediate release core and coating wherein said coating comprises modified release material and other pharmaceutical excipients.

Modified release materials comprise, but are not limited to: polyvinylpyrrolidone (K90), Hydroxypropylmethylcellulose (K4M, K10), hydroxypropylcellulose (high viscosity grade), carnauba wax, glyceryl behenate, castor wax, polyvinyl acetate, carboxymethyl ethyl cellulose, ethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide and the like. Particular modified release materials include polyvinylpyrrolidone (K90), hydroxypropylmethylcellulose (K4M, K10), hydroxypropylcellulose (high viscosity grade-HF), polyethylene oxide and the like. A modified release material may conveniently be present in the range of 10-50% wt. of the tablet.

An exemplary modified release tablet of Talabostat or a pharmaceutically acceptable salt thereof is given below in Table 3:

TABLE 3

| Formulation content | Amount (w/w %) |
|---|---|
| Talabostat as an API (available as | 0.01-2 |
| Talabostat mesylate) | |
| Polyvinylpyrrolidone (K90) or | 10-50 |
| hydroxypropylmethylcellulose (K4M, | |
| K10) or hydroxypropylcellulose (high | |
| viscosity grade-HF) or polyethylene oxide | |
| as a modified release material | |
| Sodium starch glycolate or crospovidone | 0-10 |
| as a disintegrant | |
| Magnesium stearate or stearic acid as a | 0.1-10 |
| lubricant | |
| Citric acid or phosphoric acid as a pH | 0-15 |
| modifying agent | |
| Lactose as a filler | 30-90 |

In some preferred embodiments, the amount of Talabostat in a unit dose is about 50 micrograms, about 100 micrograms per tablet, about 200 micrograms per tablet, about 300 micrograms per tablet, about 400 micrograms per tablet, about 500 micrograms per tablet, about 600 micrograms per tablet, about 700 micrograms per tablet, about 800 micrograms per tablet.

Various methods can be used for manufacturing tablets of Talabostat or a pharmaceutically acceptable salt thereof for use in the treatment regimen of this disclosure. One process includes dissolving Talabostat in a suitable solvent (with or without binder) and this solution is distributed uniformly over filler particles (which may contain other materials) to form agglomerated particles/granules. Wet granulation, coating or spraying processes can also be used. Granules may be appropriately sized or may be further processed by a dry granulation/slugging/roller compaction method followed by a milling step to achieve suitable granules of specific particle size distribution. The sized granules may be further blended with other components and/or and then lubricated in a suitable blender and compressed into tablets of specific dimensions using appropriate tooling. Coating of the tablets, where appropriate, may be performed using conventional methods and standard equipment.

### 5. KITS

In some embodiments, the kit includes a formulation comprising Talabostat or pharmaceutically acceptable salt thereof and a formulation comprising Pembrolizumab with or without instructions for their use. The combined therapeutics can be manufactured and/or formulated by the same or different manufacturers. The combination therapeutics may thus be entirely separate pharmaceutical dosage forms or pharmaceutical compositions that are also sold independently of each other. In embodiments, instructions for their combined use are provided: (i) prior to release to physicians (e.g. in the case of a “kit of part” comprising a first therapeutic agent and the other therapeutic agent); (ii) by the physicians themselves (or under the guidance of a physician) shortly before administration; (iii) the patient themselves by a physician or medical staff

In one example, a single bolus dose may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the disclosure are dictated by and directly dependent on the unique characteristics of the active compound and the particular therapeutic or prophylactic effect to be achieved.

In some embodiments, the kit comprises a package insert comprising instructions for using Talabostat or pharmaceutically acceptable salt thereof and Pembrolizumab to treat or delay progression of cancer in a subject or to enhance immune function of a subject having cancer. In some embodiments, the kit comprises Talabostat or pharmaceutically acceptable salt thereof and Pembrolizumab and package insert comprising instructions for using the same to treat or delay progression of cancer in a subject or to enhance immune function of a subject having cancer. In some embodiments, the kit comprises Talabostat or pharmaceutically acceptable salt thereof and Pembrolizumab, and a package insert comprising instructions for using the same to treat or delay progression of cancer in a subject or to enhance immune function of a subject having cancer.

In some embodiments, the kit comprises a container that includes, but is not limited to bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in a subject or for enhancing immune function of a subject having cancer. The kit may further comprise other materials desirable from a commercial and user standpoint, including other buffers, diluents, filters, needles, and syringes.

### 6. OUTCOMES

Patients afflicted with prostate cancer (e.g. SCNC) administered Talabostat or a pharmaceutically acceptable salt thereof and Pembrolizumab according to the treatment regimen disclosed herein preferably experience improvement in at least one sign of cancer. In one embodiment, improvement may be measured by a reduction in the quantity and/or size of measurable tumor lesions. In another embodiment, lesions can be measured using x-rays or CT or MRI scans. In another embodiment, cytology or histology can be used to evaluate responsiveness to the therapy. In another embodiment, extension of progression free survival and/or overall survival may be provided to a patient afflicted with prostate cancer (e.g. SCNC). In another embodiment, extension of progression free survival and/or overall survival may be provided to a patient afflicted with advanced solid cancer

In specific aspects, the anti-tumor response is a tumor specific response, a clinical response, a decrease in tumor size/volume, a decrease in tumor specific biomarkers, increase in anti-tumor cytokines or a combination thereof.

In a specific aspect, the clinical response is a decreased tumor growth and/or a decrease in tumor size. In a specific aspect, the initiating, sustaining or enhancing an anti-tumor immune response is for the treatment of cancer.

In a further aspect, the anti-tumor response is inhibiting tumor growth, inducing tumor cell death, tumor regression, preventing or delaying tumor recurrence, tumor growth, tumor spread or tumor elimination.

In a further aspect, the anti-tumor response is reduction in metastasis, delay in metastasis or prevention of metastasis. In preferred embodiment, the anti-tumor response is prevention of metastasis.

In specific embodiments, the tumor response is a decrease in the number of tumor cells. In specific embodiments, the tumor response is a decreased rate in tumor growth. In specific embodiments, the tumor response is a block in the dipeptidyl peptidase enzyme activity. In specific embodiments, the tumor response is an induction of proinflammatory cytokine response and a cytotoxic T cell response.

The treatment regimen described herein may result in an inhibition of tumor size more than about 10%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than 66%, more than 67%, more than 68%, more than 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than more than about 90%, more than 91%, more than 92%, more than 93%, more than 94%, more than about 95%, more than 96%, more than 97%, more than 98%, more than 99% up to about 100%.

In some embodiments, the regimen and methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

In some embodiments, the regimen or methods provided herein can provide for a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the risk of developing a metastasis or the risk of developing an additional metastasis in a patient having prostate cancer.

In some embodiments, the treatment regimen or methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In one embodiment, patients afflicted with prostate cancer (e.g. SCNC) administered Talabostat or a pharmaceutically acceptable salt thereof and Pembrolizumab according to the treatment regimen disclosed herein may exhibit a complete response (CR), a partial response (PR), stable disease (SD), immune-related complete disease (irCR), immune-related partial response (irPR), or immune-related stable disease (irSD). In another embodiment, patients afflicted with advanced solid cancer administered Talabostat or a pharmaceutically acceptable salt thereof and Pembrolizumab according to the treatment regimen disclosed herein may exhibit a complete response (CR), a partial response (PR), stable disease (SD), immune-related complete disease (irCR), immune-related partial response (irPR), or immune-related stable disease (irSD).

In another embodiment, patients afflicted with prostate cancer (e.g. SCNC) administered Talabostat or a pharmaceutically acceptable salt thereof and Pembrolizumab according to the treatment regimen disclosed herein may experience tumor shrinkage and/or decrease in growth rate, i.e., suppression of tumor growth. In another embodiment, unwanted cell proliferation may be reduced or inhibited.

In yet another embodiment, one or more of the following may occur in patients afflicted with prostate cancer (e.g. SCNC) administered Talabostat or a pharmaceutically acceptable salt thereof and Pembrolizumab according to the treatment regimen disclosed herein: the number of cancer cells may be reduced; tumor size may be reduced; cancer cell infiltration into peripheral organs may be inhibited, retarded, slowed, or stopped; tumor metastasis may be slowed or inhibited; tumor growth may be inhibited; recurrence of tumor may be prevented or delayed; one or more of the symptoms associated with cancer may be alleviated.

In other embodiments, patients afflicted with prostate cancer (e.g. SCNC) administered Talabostat or a pharmaceutically acceptable salt thereof and Pembrolizumab according to the treatment regimen disclosed herein may exhibit at least one therapeutic effect selected from the group consisting of reduction in size of a tumor, reduction in number of metastatic lesions appearing over time, complete remission, partial remission, or stable disease.

In still other embodiments, the treatment regimen herein may produce a comparable clinical benefit rate (CBR=CR+PR+SD≥6 months) better than that achieved by Talabostat or a pharmaceutically acceptable salt thereof, or Pembrolizumab alone.

In other embodiments, the improvement of clinical benefit rate achieved using the treatment regimen of the present disclosure may be about 20%, 30%, 40%, 50%, 60%, 70%, 80% or more compared to treatment using Talabostat or Pembrolizumab alone.

In some embodiments, the treatment regimen of the present disclosure may result in the CD8+ T cells in the subject having enhanced priming, activation, proliferation and/or cytolytic activity when compared to administration of Talabostat or a pharmaceutically acceptable salt thereof, or Pembrolizumab alone.

In some embodiments, the number of CD4+ and/or CD8+ T cells is elevated relative to prior to administration of the combination.

In some embodiments, the activated CD4+ and/or CD8+ T cells is characterized by y-IFN+ producing CD4+ and/or CD8+ T cells and/or enhanced cytolytic activity relative to prior to the administration of the combination.

In some embodiments, the CD4+ and/or CD8+ T cells exhibit increased release of cytokines selected from the group consisting of G-CSF, MCP-1, Eotaxin, IFN-γ, KC, TNF-α and interleukins (IL-5, IL-6, IL-1β, IL-12p70, IL 18).

In some embodiments, the CD4+ and/or CD8+ T cell is an effector memory T cell. In some embodiments, the CD4+ and/or CD8+ effector memory T cell is characterized by γ-IFN+ producing CD4+ and/or CD8+ T cells and/or enhanced cytolytic activity.

In some embodiments, the serum levels of cytokine IL-18 and/or chemokine GM-CSF, G-CSF in the subject are increased in the presence of combination of Talabostat or a pharmaceutically acceptable salt thereof and Pembrolizumab as compared to single agent administration.

In some embodiments, the cancer has elevated levels of T-cell infiltration when a combination of Talabostat or a pharmaceutically acceptable salt thereof, and Pembrolizumab is used according to the treatment regimen described herein, when compared to administration of Talabostat or Pembrolizumab alone.

In some embodiments, the cancer has suppressed/decreased levels of T-regulatory cells in the presence of a combination of Talabostat or a pharmaceutically acceptable salt thereof and Pembrolizumab when used according to the treatment regimen described herein, when compared to administration of Talabostat or a pharmaceutically acceptable salt thereof, or Pembrolizumab alone. In some embodiments, the cancer has increased levels of NK cells and macrophages in the presence of combination of Talabostat or a pharmaceutically acceptable salt thereof and Pembrolizumab when used according to the treatment regimen described herein, when compared to administration of Talabostat or a pharmaceutically acceptable salt thereof, or Pembrolizumab alone.

With respect to target lesions, responses to the treatment regimen described herein may include: Complete response (CR), Partial Response (PR), Progressive Disease (PD), Stable Disease (SD), Immune-related Complete Response (irCR), Immune-related Partial Response (irPR), Immune-related Progressive Disease (irPD) and Immune-related Stable Disease (irSD).

With respect to non-target lesions, responses to the treatment regimen described herein may include: Complete Response (CR), Progressive Disease (PD), Immune-related Complete Response (irCR) and Immune-related Progressive Disease (irPD).

In one embodiment, the patient treated exhibits a complete response (CR), a partial response (PR), stable disease (SD), immune-related complete disease (irCR), immune-related partial response (irPR), or immune-related stable disease (irSD). In another embodiment, the patient treated experiences tumor shrinkage and/or decrease in growth rate, i.e., suppression of tumor growth. In another embodiment, unwanted cell proliferation is reduced or inhibited. In yet another embodiment, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

### 7. SPECIFIC EMBODIMENTS OF THE PRESENT DISCLOSURE

### Embodiment 1

A treatment regimen for treating prostate cancer in a subject in need thereof, comprising administering to said subject, as separate pharmaceutical formulations, an effective amount of Talabostat or a pharmaceutically acceptable salt thereof and an effective amount of Pembrolizumab.

### Embodiment 2

A method of treating prostate cancer in a subject in need thereof, the method comprising administering to the subject, as separate pharmaceutical formulations, an effective amount of Talabostat or a pharmaceutically acceptable salt thereof and an effective amount of Pembrolizumab.

### Embodiment 3

A method of enhancing an immune response in a subject suffering from prostate cancer, the method comprising administering to said subject a regimen comprising, as separate pharmaceutical formulations, an effective amount of Talabostat or a pharmaceutically acceptable salt thereof and an effective amount of Pembrolizumab

### Embodiment 4

A method of enhancing an innate immune response in a subject afflicted with prostate cancer, the method comprising administering to said subject, as separate pharmaceutical formulations, an effective amount of Talabostat or a pharmaceutically acceptable salt thereof and an effective amount of Pembrolizumab, wherein the enhanced innate immune response is associated with increased tumoricidal natural killer cells and macrophages, as well as the activity of NK cells and CD8+ T cells.

### Embodiment 5

A method of enhancing an innate immune response in a subject with prostate cancer, the method comprising administering to said subject, as separate pharmaceutical formulations, an effective amount of Talabostat or a pharmaceutically acceptable salt thereof and an effective amount of Pembrolizumab, wherein the enhanced innate immune response is associated with suppression of T-regulatory cells

### Embodiment 6

A method for treating, delaying progression or preventing or delaying tumor recurrence, tumor growth or spread of tumor in a subject afflicted with prostate cancer (e.g. SCNC), the method comprising administering to the subject, as separate pharmaceutical formulations, effective amounts of Talabostat or a pharmaceutically acceptable salt thereof and Pembrolizumab

### Embodiment 7

A method of enhancing immune function in a subject afflicted with prostate cancer (e.g. SCNC), the method comprising administering to the subject, as separate pharmaceutical formulations, effective amounts of Talabostat or a pharmaceutically acceptable salt thereof and Pembrolizumab.

### Embodiment 8

A method for initiating, sustaining or enhancing an anti-tumor immune response in a subject afflicted with prostate cancer (e.g. SCNC), the method comprising administering to the subject, as separate pharmaceutical formulations, effective amounts of Talabostat or a pharmaceutically acceptable salt thereof and Pembrolizumab.

### Embodiment 9

A method for reducing the toxicity of Talabostat with a lower effective dose of Talabostat, the method comprising administering to a subject afflicted with prostate cancer (e.g. SCNC), as separate pharmaceutical formulations, effective amounts of Talabostat or a pharmaceutically acceptable salt thereof and Pembrolizumab.

### Embodiment 10

The treatment regimen or method of treatment according to any of Embodiments 1-9, wherein Talabostat or a pharmaceutically acceptable salt thereof and Pembrolizumab are administered to the subject in one or more (e.g. 1, 2, 3, 4, 5, 6 or more) treatment cycles, where each treatment cycle is of about 21 days duration.

### Embodiment 11

The treatment regimen or method of treatment according to any of Embodiments 1-10, wherein after cessation of treatment the subject maintains a sustained response to progression of prostate cancer.

### Embodiment 12

The treatment regimen or method of treatment according to Embodiment 10 or 11, wherein for each treatment cycle Talabostat or a pharmaceutically acceptable salt thereof is administered on each of days 1 to 14 and Pembrolizumab is administered on day 1.

### Embodiment 13

The treatment regimen or method of treatment according to any of Embodiments 1-12, wherein Talabostat or a pharmaceutically acceptable salt thereof is administered orally (e.g. as a tablet formulation).

### Embodiment 14

The treatment regimen or method of treatment according to any of Embodiments 1-12, wherein Pembrolizumab is administered by injection (e.g. intravenously).

### Embodiment 15

The treatment regimen or method of treatment according to any of Embodiments 1-14, wherein Talabostat or a pharmaceutically acceptable salt thereof is administered at a total daily dose of from about 0.001 mg/kg to about 0.1 mg/kg (e.g. about 0.001 mg/kg to about 0.035 mg/kg, or about 0.001 mg/kg to about 0.014 mg/kg).

### Embodiment 16

The treatment regimen or method of treatment according to any of Embodiments 1-15, wherein Pembrolizumab is administered at a dose of from about 1 mg/kg to about 10 mg/kg per day.

### Embodiment 17

The treatment regimen or method of treatment according to any of Embodiments 1-16, wherein Talabostat or a pharmaceutically acceptable salt thereof is administered at a total daily dose of from about 0.4 mg to about 0.6 mg (e.g. administered orally twice daily, such as at a dose of about 0.4 mg in the morning and about 0.2 mg in the evening).

### Embodiment 18

The treatment regimen or method of treatment according to any of Embodiments 1-17, wherein Pembrolizumab is administered at a total dose of from about 100 mg to about 500 mg per day (e.g. about 200 mg per day).

### Embodiment 19

The treatment regimen or method of treatment according to any of Embodiments 1-18, wherein the total daily dose of Talabostat or a pharmaceutically acceptable salt thereof in cycle 1 is lower than the total daily dose of Talabostat or a pharmaceutically acceptable salt thereof in one or more subsequent cycles.

### Embodiment 20

The treatment regimen or method of treatment according to any of Embodiments 1-19, comprising administering Talabostat mesylate.

### Embodiment 21

A treatment regimen for treating prostate cancer in a subject in need thereof, the regimen comprising administering to the subject Talabostat mesylate and Pembrolizumab in one or more treatment cycles, where each treatment cycle is of about 21 days duration, and for each treatment cycle Talabostat is administered on each of days 1 to 14 and Pembrolizumab is administered on day 1, wherein Talabostat mesylate is administered as one or more tablets to provide a total daily dose of Talabostat of from about 0.4 mg to about 0.6 mg and Pembrolizumab is administered as a single intravenous injection to provide a dose of from about 100 mg to about 500 mg per day.

### 8. EXAMPLES

### Example 1

### Treatment Regimen for a Phase 1b/2 Study

This Phase 1b/2 study is to determine the composite response rate of a pharmaceutical formulation of Talabostat mesylate administered orally and daily, combined with a pharmaceutical formulation of Pembrolizumab (more specifically Keytruda®) in patients with SCNC. The study will also assess other efficacy parameters, such as rPFS, PSA, PFS, OS and DOR, as well as the safety of the combined treatment. The study will consist of two stages: Lead-in Stage—in which the safety and tolerability of the combination of Talabostat mesylate administered once daily (QD) on Days 1 to 14 of a 21-day cycle plus Pembrolizumab 200 mg administered intravenously (IV) on Day 1 every 21 days was assessed. During the Lead-in Stage, the dose of Pembrolizumab was fixed (200 mg IV q21 days) with doses of Talabostat mesylate being escalated (0.4 mg to 0.6 mg per oral QD days 1-14 of 21-day cycles) using a 3×3 design and confirmed in patients with SCNC. In Cycle 1, the initial dose of Talabostat mesylate administered was 0.4 mg. As there were no safety concerns following treatment, the dose was escalated to 0.6 mg. There was a 7-day rest period following the last (14th) dose of Talabostat mesylate and Day 1 of the subsequent cycle. The key endpoints were safety and identification of the recommended phase 2 dose (RP2D) for the combination. Composite responses (RECIST, PSA, CTC) were also assessed. If there are no safety concerns at 0.6 mg then this is the RP2D to be used in the Efficacy stage. If there are safety concerns, the 0.6 mg cohort will be expanded to enroll additional patients. The dosing schedule may also be adjusted.

During the Lead-in Stage, patients were observed for dose-limiting toxicity (DLT) during Cycle 1. Three patients were treated initially with 0.4 mg Talabostat mesylate plus Pembrolizumab (200 mg IV):
There were no DLTs in Cycle 1 so the dose of Talabostat mesylate was escalated to 0.6 mg in the next cohort of 3 patients.
If ≥1 of the 3 original patients has a DLT in Cycle 1, after a discussion between the sponsor and the investigator, either 3 patients (if 1 patient experiences a DLT) or 6 to 9 patients (if 2 or 3 patients experiences a DLT) will be added at the 0.4 mg Talabostat mesylate dose level. For this expanded 0.4 mg cohort:
If less than one-third of the patients experience a DLT, consideration will be given to dose escalation to 0.6 mg Talabostat mesylate plus Pembrolizumab
If one-third of the patients experience a DLT, the Efficacy Stage can commence
If more than one-third of the patients experience a DLT, a discussion will be held between the investigators and sponsors as to how to proceed.

Following dose escalation to 0.6 mg Talabostat mesylate plus Pembrolizumab in 3 patients:
If there are no DLTs or ⅓ patients has a DLT at this dose level, the Efficacy Stage can commence.
If ≥⅓ patients have a DLT in Cycle 1, after a discussion between the sponsor and the investigator, 6 to 9 patients was added at the 0.4 mg Talabostat mesylate dose level.
If ≤⅓ patients experience a DLT, the Efficacy Stage can commence with the 0.6 mg Talabostat mesylate dose plus Pembrolizumab
If >⅓ patients experience a DLT, consideration will be given to the use of 0.4 mg Talabostat mesylate plus Pembrolizumab in the Efficacy Stage.

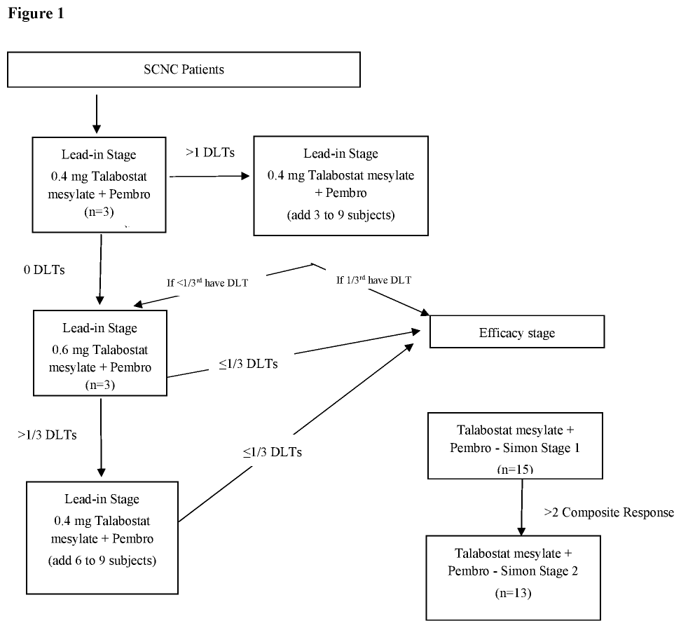

The study schema is presented in FIG. 1.

Patients in the Lead-in Stage who do not meet the criteria for discontinuation will be allowed to continue treatment at their originally assigned doses.

All safety data from all patients enrolled in each cohort will be reviewed to confirm any DLTs that were experienced and to determine enrolling the next cohort, as well as the Talabostat mesylate dose to be used in the Efficacy Stage. Unless doses are held because of AEs, a patient must have received >70% of their Talabostat mesylate doses in Cycle 1 (i.e., ≥10 of 14 planned doses) with Pembrolizumab dosed on Day 1 of Cycle 1 to be eligible for DLT assessment.

Toxicities will be assessed by the investigator using the National Cancer Institute Common Terminology Criteria for Adverse Events (NCI CTCAE), version 5. The relationship of an AE to combination therapy (i.e., attribution to Talabostat mesylate and/or Pembrolizumab) is to be assessed by the investigator using the criteria in the protocol.

A DLT is defined as any of the following AEs occurring during Cycle 1, regardless of investigator attribution to study treatment, unless the AE can be clearly and incontrovertibly attributed to an extraneous cause (e.g., disease progression):
Any Grade 4 laboratory abnormality, regardless of duration
Any Grade 3 non-hematologic AE, with the exceptions of Grade 3 nausea, vomiting, diarrhea, constipation, fever, fatigue, skin rash, or non-clinically significant laboratory abnormality that resolves to Grade ≤2 within 72 hours with optimal medical management.
Grade 3 thrombocytopenia with Grade >1 bleeding or requirement for platelet transfusion.
Grade 3 febrile neutropenia.
Grade 3 fever.
Grade 3 skin rash.
Laboratory abnormalities meeting Hy's law criteria (aspartate aminotransferase [AST] or alanine aminotransferase [ALT] >3×upper limit of normal [ULN] with concomitant total bilirubin >2×ULN).
Grade 3 transaminase (AST/ALT) elevation.
Any toxicity resulting in ≥30% held/skipped doses of Talabostat mesylate during Cycle 1.
Delay of Cycle 2 by ≥14 days due to toxicity.
Any other significant toxicity considered by the investigator and sponsor's medical representatives to be dose-limiting.

### Efficacy Stage:

After assessment of the safety and confirmation of the Talabostat mesylate/Pembrolizumab dose schedule (i.e., either total daily dose of 0.6 mg or 0.4 mg Talabostat mesylate) to be used in the subsequent stage, the Efficacy Stage will begin. Eligible SCNC patients will receive Talabostat mesylate QD on Days 1 to 14 of a 21-day cycle plus Pembrolizumab 200 mg administered IV on Day 1 every 21 days.

Study Design Features (Both Stages):

In the Lead-in Stage, patients were screened for study eligibility within 28 days before the first study drug dose after provision of written informed consent. Patients who were determined to be eligible, based on Screening assessments, were enrolled in the study on Cycle 1, Day 1 (Baseline, before the first dose of Talabostat mesylate). Similarly, in the Efficacy Stage, patients will be screened as per the above process.

During treatment, patients will attend study center visits and have study evaluations performed as detailed in the Schedule of Assessments. All study visits will be conducted on an outpatient basis but may be conducted on an inpatient basis per the investigator's judgement.

All patients must have pre-treatment (prior to study treatment dosing) imaging (computed tomography [CT] scan of chest/abdomen/pelvis or magnetic resonance imaging [MRI] for baseline tumor measurements, as well as bone scintigraphy [BS]). Patients with skin, subcutaneous or lymph node metastases may also have tumor evaluations (including measurements, with a ruler) by means of physical examination. Patients with a history of central nervous system (CNS) malignant involvement or CNS symptoms should have either CT or MRI imaging of the brain performed to assess active CNS malignancy.

Tumor measurements and disease response assessments (CT or MRI; BS) are also to be performed at the end of Cycle 3 (approximately 9 weeks after the first study treatment dose), and then approximately every 9 weeks thereafter until development of progressive disease (PD). For patients with evidence of disease control (stable disease or better) at Week 27, tumor measurements and disease response assessments may be performed less frequently (approximately every 12 weeks) thereafter. Tumor measurements and disease response assessments also are to be performed at the End of Treatment (EOT) visit.

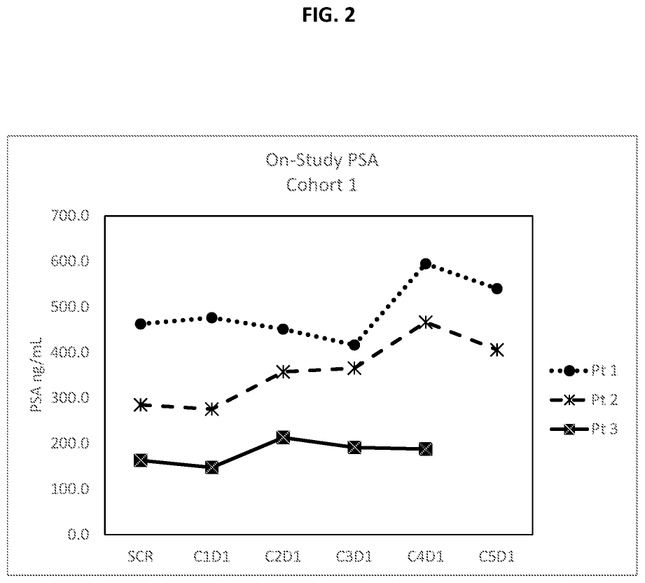

Additionally, measurement of serum PSA will be performed on Day 1 of every treatment cycle. See FIG. 2.

Enumeration of CTCs by Veridex assay will be performed on Cycle 1 Day 1, Cycle 2 Day 1, Cycle 4 Day 1, and every 3 cycles thereafter until EOT visit.

Population pharmacokinetics of Talabostat mesylate will be assessed using sparse pharmacokinetic sampling.

Patients may continue to receive treatment until the development of radiographic progression by RECIST 1.1/Prostate Cancer Working Group 3 (PCWG3) criteria, unequivocal clinical progression, unacceptable toxicity, another discontinuation criterion is met, or closure of the study; no maximum duration of therapy has been set. Patients with PSA progression in the absence of radiographic or clinical progression should continue to receive protocol therapy.

### Study Population

Approximately 6 to 12 patients with SCNC who fulfilled the eligibility criteria of the protocol were enrolled during the Lead-in Stage. Approximately 15 to 28 patients who fulfill the eligibility criteria of the protocol will be enrolled during Efficacy Stage.

### Eligibility Criteria

All patients must satisfy the following inclusion and exclusion criteria to be eligible for entry into the trial. For patients with histologic evidence of SCNC on archival tissue analysis, enrollment can proceed without obtaining pathology review of fresh biopsy.

### Inclusion Criteria

1. Patient has evidence of progressive, metastatic castration-resistant disease, as defined by PCWG3 criteria.

a. Patients with de novo small cell prostate cancer are not required to have received androgen deprivation therapy (ADT).

2. Progression during or following completion of at least 1 prior line of systemic therapy for locally advanced or metastatic prostate cancer.
3. Efficacy Stage only:

a. Patient has histologic evidence of SCNC on central pathology review of archival tumor tissue. Patients without evaluable archival metastatic tumor tissue may undergo fresh tumor biopsy during Screening.

b. Must be willing to undergo metastatic tumor biopsy during Screening. Requirement may be waived in patients without safely accessible lesion or for patients with evaluable archival metastatic tumor tissue

c. Patient has previously received at least 1 prior line of cytotoxic chemotherapy. Patients who either have refused chemotherapy or considered unsuitable for chemotherapy may be eligible following discussion with the sponsor.

4. Patient has serum testosterone <50 ng/dL during Screening except for those with de novo small cell prostate cancer.

a. Patients with treatment-emergent SCNC without a history of bilateral orchiectomy are required to remain on luteinizing hormone-releasing hormone (LHRH) analog during the course of protocol therapy except for patients with de novo small cell prostate cancer.

5. Patient has Eastern Cooperative Oncology Group (ECOG) performance status of 0-2.
6. Patient is aged ≥18 years.
7. Patient's acute toxic effects of previous anticancer therapy have resolved to ≤Grade 1 except for Grade 2-3 peripheral neuropathy or any grade of alopecia.
8. Patient has adequate baseline organ function, as demonstrated by the following:

a. Serum creatinine ≤1.5 times institutional ULN or calculated creatinine clearance ≥50 mL/min;

b. Serum albumin ≥2.5 g/dL;

c. Total bilirubin ≤1.5×ULN;

d. AST and ALT ≤2.5×institutional ULN (patients with hepatic metastases must have AST/ALT ≤5×ULN);

9. Patient has adequate baseline hematologic function, as demonstrated by the following:

a. Absolute neutrophil count (ANC) ≥1.5×109/L.

b. Hemoglobin ≥8 g/dL and no red blood cell transfusions during the prior 14 days.

c. Platelet count ≥100×109/L and no platelet transfusions during the prior 14 days.

10. Patient agrees to use acceptable contraceptive methods for the duration of time on the study and continue to use acceptable contraceptive methods for 6 months after the last treatment dose.
11. Patient has signed informed consent prior to initiation of any study-specific procedures or treatment.
12. Patient is able to adhere to the study visit schedule and other protocol requirements, including follow-up for OS.

### Exclusion Criteria

1. Patient has received treatment with >2 cytotoxic chemotherapy regimens for CRPC. Chemotherapy in the hormone-sensitive setting does not count in this assessment provided the last dose was >6 months before study entry.
2. Patient has received external-beam radiation or another systemic anticancer therapy within 14 days or 5 half-lives, whichever is shorter, prior to study treatment.
3. Patient has received treatment with an investigational systemic anticancer agent within 14 days prior to study drug administration.
4. Patient has received prior treatment with an anti-PD-1, anti-PD-L1, anti-programmed death-ligand 2 (PD-L2) agent or with an agent directed to another co-inhibitory T-cell receptor (e.g., cytotoxic T-lymphocyte-associated antigen 4 [CTLA-4], OX-40, CD137, sipuleucel-T).
5. Patient has an additional active malignancy that may confound the assessment of the study endpoints. If the patient has a past cancer history (active malignancy within 2 years prior to study entry) with substantial potential for recurrence, this must be discussed with the sponsor before study entry. Patients with the following concomitant neoplastic diagnoses are eligible: non-melanoma skin cancer and carcinoma in situ (including transitional cell carcinoma, anal carcinoma, and melanoma in situ).
6. Patient has clinically significant cardiovascular disease (e.g., uncontrolled or any New York Heart Association Class 3 or 4 congestive heart failure, uncontrolled angina, history of myocardial infarction, unstable angina or stroke within 6 months prior to study entry, uncontrolled hypertension or clinically significant arrhythmias not controlled by medication).
7. QT interval corrected for heart rate using Bazett's formula (QTcB) >440 msec at Screening.
8. Patient has uncontrolled, clinically significant pulmonary disease (e.g., chronic obstructive pulmonary disease, pulmonary hypertension) that in the opinion of the investigator would put the patient at significant risk for pulmonary complications during the study.
9. Patient has brain or leptomeningeal metastases that are symptomatic and progressive on imaging. Patients with a history of CNS metastases must have received appropriate treatment. CNS imaging is not required prior to study entry unless there is a history of CNS involvement or clinical suspicion of CNS involvement. Imaging of patients with a prior history of CNS metastases should be compared to prior imaging to discern disease progression.
10. Patient has an active autoimmune disease or Grade ≥3 pneumonitis that has required systemic treatment in the past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) or treatment with drugs (e.g., neomercazol, carbimazole, etc.) that function to decrease the generation of thyroid hormone by a hyperfunctioning thyroid gland (e.g., in Graves' disease) is not considered a form of systemic treatment of an autoimmune disease.
11. Patient has a diagnosis of immunodeficiency or is receiving systemic steroid therapy at a prednisone equivalent dose of >10 mg daily for at least 1 week or other form of immunosuppressive therapy within 7 days prior to C1D1.
12. Patient has uncontrolled intercurrent illness including, but not limited to, uncontrolled infection, disseminated intravascular coagulation, or psychiatric illness/social situations that would limit compliance with study requirements.
13. Patient has known positive status for human immunodeficiency virus active or chronic Hepatitis B or Hepatitis C. Screening is not required.
14. Patient has any medical condition which in the opinion of the investigator places the patient at an unacceptably high risk for toxicity.

### Study Methodology

Approximately 6 to 12 patients with SCNC were enrolled in the Lead-in Stage of the study.

Approximately 15 to 28 patients with SCNC will be enrolled in the Efficacy Stage of the study. In the first stage, 15 patients will be accrued. If there are 2 or fewer composite responses in these 15 patients, accrual to the study arm will be halted. Otherwise, 13 additional patients will be accrued for a total of 28 patients. A composite response is defined as 1 or more of the following:
Objective response by RECIST 1.1 criteria
CTC conversion from >5/7.5 mL to <5/7.5 mL per Veridex assay by completion of Week 12 of protocol therapy
Greater than 50% PSA decline from baseline by completion of Week 12 of protocol therapy

The tabular Study Schedule of Assessments is found in Appendix A. The description of Detailed Study Procedures by visit is found in Appendix B.

During the Screening period, patients who have signed a consent form will be evaluated to ensure they meet inclusion and exclusion criteria. Patient demographics, performance status, and disease staging will be collected. Vital signs (both sitting and standing blood pressure, heart rate, body temperature, and respiratory rate), physical examination, electrocardiogram (ECG), and clinical laboratory evaluations (complete blood count plus differential, serum chemistry, liver function tests, and urinalysis) will be performed at Screening and baseline, and will be monitored throughout the treatment period. Patients meeting the study entry criteria will begin study treatment within 4 weeks of the Screening visit. At the discretion of the investigator, patients with out-of-range clinical laboratory evaluation values at Screening may be retested within the Screening period if the investigator believes that the retest values may be in range, and allow inclusion in the study.

Patients will undergo tumor assessment which must include cross-sectional imaging (MRI or CT scanning with IV contrast whenever possible) of the chest/abdomen/pelvis plus whole-body bone scan. Other body sites (e.g., neck) to be included as clinically indicated. Tumor assessment will be performed at Screening, C4D1 (±7 days), C7D1 (±7 days), C10D1 (±7 days), and Day 1 (±7 days) of every 3rd cycle thereafter.

The same imaging method used to determine index lesion size at baseline must be used to follow lesion size throughout the study. Assessments will be conducted throughout the treatment phase as described in Appendix A and Appendix B.

AEs, clinical laboratories, PSA, and concomitant medications will be monitored and recorded throughout the entire study period.

Patients may continue to receive study treatment until the development of radiographic or clinical progression, unacceptable toxicity, another discontinuation criterion is met, or closure of the study by the sponsor; no maximum duration of therapy has been set.

After discontinuation of study treatment, patients will complete an EOT visit within 21 days after their last dose of study drug. Safety Follow-up is to be conducted 30 days (±7 days) after their last dose of study drug as well as additional subsequent time points if drug-related AEs have not resolved at that time. Patients will also be contacted by telephone approximately every 90 days for clinical evidence of disease progression in settings in which discontinuation of study therapy was for reasons other than PD (tumor measurements as specified in the protocol are not required after the EOT visit), and for assessment of survival status. This extended follow-up for disease status and survival after discontinuation of study treatment will continue for up to 12 months after study treatment was started.

### Concomitant Medications

Permitted Medications/Therapies:

All medications taken within 3 months of study start and used throughout the study must be recorded in the appropriate section of the patient's electronic case report form (eCRF).

Patients who have not had prior bilateral orchiectomy must continue LHRH analogue treatment to maintain castrate level of testosterone during the course of study treatment (except for patients with de novo small cell prostate cancer).

The use of bone-modifying agents (e.g., zoledronic acid, denosumab) is permitted as clinically indicated.

The use of growth factors (e.g., granulocyte-colony stimulating factor [G-CSF]) is allowed as clinically indicated for the treatment of Grade ≥3 cytopenias.

The use of any other systemic anti-cancer therapies other than outlined in the protocol is prohibited during the course of study treatment.

Suggested supportive care measures for the management of AEs with potential immunologic etiology are outlined below. Where appropriate, these guidelines include the use of oral or IV treatment with corticosteroids as well as additional anti-inflammatory agents if symptoms do not improve with administration of corticosteroids. Note that several courses of steroid tapering may be necessary as symptoms may worsen when the steroid dose is decreased. For each AE, attempts should be made to rule out other causes such as metastatic disease or bacterial or viral infection, which might require additional supportive care. The treatment guidelines are intended to be applied when the investigator determines the events to be related to Pembrolizumab.

It may be necessary to perform conditional procedures such as bronchoscopy, endoscopy, or skin photography as part of the evaluation of the event.
Pneumonitis:
For Grade 2 events, treat with systemic corticosteroids (e.g., oral prednisone 1 mg/kg or equivalent). When symptoms improve to Grade 1 or less, steroid taper should be started and continued over no less than 4 weeks.
For Grade 3-4 events, immediately treat with IV steroids (e.g., solumedrol 1-2 mg/kg every 6-8 hours). Administer additional anti-inflammatory measures, as needed. When symptoms improve to Grade 1 or less, steroid taper should be started and continued over no less than 4 weeks.
Add prophylactic antibiotics for opportunistic infections in the case of prolonged steroid administration.
Diarrhea/Colitis:
   Patients should be carefully monitored for signs and symptoms of enterocolitis (such as diarrhea, abdominal pain, blood or mucus in stool, with or without fever) and of bowel perforation (such as peritoneal signs and ileus).
All patients who experience diarrhea/colitis should be advised to drink liberal quantities of clear fluids. If sufficient oral fluid intake is not feasible, fluid and electrolytes should be administered via IV infusion. For Grade 2 or higher diarrhea, consider gastroenterology consultation and endoscopy to confirm or rule out colitis.
For Grade 2 diarrhea/colitis, administer oral corticosteroids (e.g., oral prednisone 1 mg/kg or equivalent). When symptoms improve to Grade 1 or less, steroid taper should be started and continued over no less than 4 weeks.
For Grade 3 or 4 diarrhea/colitis, treat with IV steroids (e.g., solumedrol 1-2 mg/kg every 6-8 hours) followed by high dose oral steroids. When symptoms improve to Grade 1 or less, steroid taper should be started and continued over no less than 4 weeks.
Type 1 diabetes mellitus (T1DM) (if new onset, including diabetic ketoacidosis [DKA]) or ≥Grade 3 Hyperglycemia, if associated with ketosis (ketonuria) or metabolic acidosis (DKA)
For T1DM or Grade 3-4 Hyperglycemia
Insulin replacement therapy is recommended for T1DM and for Grade 3-4 hyperglycemia associated with metabolic acidosis or ketonuria.
Evaluate patients with serum glucose and a metabolic panel, urine ketones, glycosylated hemoglobin, and C-peptide.
Hypophysitis:
For Grade 2 events, treat with corticosteroids (e.g., oral prednisone 1 mg/kg/day). When symptoms improve to Grade 1 or less, steroid taper should be started and continued over no less than 4 weeks. Replacement of appropriate hormones may be required as the steroid dose is tapered.
For Grade 3-4 events, treat with an initial dose of IV corticosteroids (e.g., solumedrol 1-2 mg/kg every 6-8 hours) for 24-48 hours, followed by high dose oral corticosteroids. When symptoms improve to Grade 1 or less, steroid taper should be started and continued over no less than 4 weeks. Replacement of appropriate hormones may be required as the steroid dose is tapered.
Hyperthyroidism or Hypothyroidism:
   Thyroid disorders can occur at any time during treatment. Monitor patients for changes in thyroid function (at the start of treatment, periodically during treatment, and as indicated based on clinical evaluation) and for clinical signs and symptoms of thyroid disorders.
Grade 2 hyperthyroidism events and Grade 2-4 hypothyroidism:
In hyperthyroidism, non-selective beta-blockers (e.g., propranolol) are suggested as initial therapy.
In hypothyroidism, thyroid hormone replacement therapy, with levothyroxine or liothyronine, is indicated per standard of care.
Grade 3-4 hyperthyroidism
Treat with an initial dose of IV corticosteroids followed by oral corticosteroids. When symptoms improve to Grade 1 or less, steroid taper should be started and continued over no less than 4 weeks. Replacement of appropriate hormones may be required as the steroid dose is tapered.
Hepatic:
For Grade 2 events, monitor liver function tests more frequently until returned to baseline values (consider weekly).
Treat with IV or oral corticosteroids (e.g., prednisone 1 mg/kg/day or equivalent).
For Grade 3-4 events, treat with IV corticosteroids for 24 to 48 hours (e.g., solumedrol 1-2 mg/kg every 6-8 hours), followed by transition to oral high dose steroids (e.g., prednisone 1 mg/kg/day or equivalent).
When symptoms improve to Grade 1 or less, a steroid taper should be started and continued over no less than 4 weeks.
Renal Failure or Nephritis:
For Grade 2 events, treat with oral corticosteroids (e.g., prednisone 1 mg/kg/day).
For Grade 3-4 events, treat with IV corticosteroids (e.g., solumedrol 1-2 mg/kg every 6-8 hours) for 24-48 hours followed by transition to oral high dose steroid (e.g., prednisone 1 mg/kg/day or equivalent).
When symptoms improve to Grade 1 or less, steroid taper should be started and continued over no less than 4 weeks.
Management of Infusion Reactions: Signs and symptoms usually develop during or shortly after drug infusion and generally resolve completely within 24 hours of completion of infusion.
   Table 4 shows treatment guidelines for patients who experience an infusion reaction associated with administration of Pembrolizumab.

TABLE 4

| | | Premedication at |
|---|---|---|
| NCI CTCAE Grade | Treatment | subsequent dosing |
| Grade 1 | Increase monitoring of vital | None |
| Mild reaction; infusion | signs as medically | |
| interruption not indicated; | indicated until the patient is | |
| intervention not indicated | deemed medically stable in | |
| | the opinion of the | |
| | investigator. | |
| Grade 2 | Stop Infusion and monitor | Patient may be |
| Requires infusion interruption but | symptoms. | premedicated 1.5 hr |
| responds promptly to symptomatic | Additional appropriate | (±30 minutes) prior |
| treatment (e.g., antihistamines, | medical therapy may | to infusion of |
| NSAIDS, narcotics, IV fluids); | include but is not limited | Pembrolizumab with: |
| prophylactic medications indicated | to: | Diphenhydramine 50 |
| for ≤24 hr | IV fluids Antihistamines | mg po (or equivalent |
| | NSAIDS Acetaminophen | dose of antihistamine). |
| | Narcotics | Acetaminophen 500- |
| | Increase monitoring of | 1000 mg po (or |
| | vital signs as medically | equivalent dose of |
| | indicated until the patient | antipyretic). |
| | is deemed medically stable in | |
| | the opinion of the | |
| | investigator. | |
| | If symptoms resolve within | |
| | 1 hour of stopping drug | |
| | infusion, the infusion may | |
| | be restarted at 50% of the | |
| | original infusion rate (e.g., | |
| | from 100 mL/hr to 50 | |
| | mL/hr). | |
| | Otherwise dosing will be | |
| | held until symptoms | |
| | resolve and the patient | |
| | should be premedicated for | |
| | the next scheduled dose. | |
| Grades 3 or 4 | Stop Infusion. | No subsequent dosing |
| Grade 3: | Additional appropriate | |
| Prolonged (i.e., not rapidly | medical therapy may | |
| responsive to symptomatic | include but is not limited | |
| medication and/or brief | to: | |
| interruption of infusion); | IV fluids Antihistamines | |
| recurrence of symptoms following | NSAIDS | |
| initial improvement; | Acetaminophen Narcotics | |
| hospitalization indicated for other | Oxygen Pressors | |
| clinical sequelae (e.g., renal | Corticosteroids | |
| impairment, pulmonary infiltrates) | Epinephrine | |
| Grade 4: | Increase monitoring of vital | |
| Life-threatening; pressor or | signs as medically | |
| ventilatory support indicated | indicated until the patient is | |
| | deemed medically stable in | |
| | the opinion of the | |
| | investigator. | |
| | Hospitalization may be | |
| | indicated. | |
| | Patient is permanently | |
| | discontinued from further | |
| | trial treatment | |
| | administration. | |

Appropriate resuscitation equipment should be available in the room and a physician readily available during the period of drug administration.
IV = intravenous;
NCI CTCAE = National Cancer Institute Common Terminology Criteria for Adverse Events;
NSAID = non-steroidal anti-inflammatory drug;
po = per os (oral)

### Prohibited Medications/Therapies

Enrolled patients may not receive investigational or approved anticancer agents including cytotoxic chemotherapy agents, anticancer tyrosine kinase inhibitors, or therapeutic monoclonal antibodies.

Palliative radiation is not permitted during study enrollment unless it is being performed for an existing, nonprogressive metastasis/symptoms and involves a narrow radiation port (e.g., solitary bone lesions).

Preclinical studies have demonstrated a low potential for Talabostat mesylate to inhibit the following major human liver CYP isoenzymes: CYP1A2, CYP2C8, CYP2C9, CYP2C19, CYP2D6, and CYP3A4. Further, relevant concentrations of Talabostat mesylate did not induce CYP3A4 or CYP1A2. Therefore, there are no prohibited medications based on CYP isoenzymes.

### Efficacy Assessments

Primary Efficacy Parameter:
The primary efficacy parameter is the composite response rate defined as achieving 1 or more of the following:
Objective response by RECIST 1.1 criteria
Circulating tumor cell (CTC) conversion from >5/7.5 mL to <5/7.5 mL per Veridex assay by completion of Week 12 of protocol therapy
Greater than 50% prostate specific antigen (PSA) decline from baseline by completion of Week 12 of protocol therapy

Secondary Efficacy Parameters:
Median Radiographic progression-free survival (rPFS) when treated with the combination
Median progression-free survival (PFS) when treated with the combination
Median Overall survival (OS) when treated with the combination.
Median Duration of response (DOR) when treated with the combination.
Characterize the safety profile of the combination
Assess population pharmacokinetics of talabostat mesylate using pharmacokinetic sampling.
Assess the pharmacodynamic profile of the combination by measuring relevant effects on cytokines previously shown to be modulated by talabostat in humans.

Exploratory Efficacy Analyses:
To determine the response rate by iRECIST criteria with Talabostat mesylate in combination with Pembrolizumab.
To evaluate the quantitative and qualitative effects of Talabostat mesylate in combination with Pembrolizumab on relevant immune effector cytokines and various immunological effector cells, including neutrophils, MDSCs, dendritic cells, CAF and T-cells in blood and, whenever feasible, in tumor tissues.
To explore the predictive value of baseline PD-L1 tumor expression in metastatic tumor tissue and CTCs with subsequent clinical outcomes
To explore the relationship between baseline and on-treatment circulating tumor neoantigens and T-cell repertoire and clinical outcomes (assessed at a central laboratory).

To explore the relationship between baseline tumor mRNA immune profiling panel and clinical outcomes.

### Safety Assessments

### Non-Serious Adverse Events

Investigators should assess for AEs at each visit. All AEs, including observed or volunteered problems, complaints, or symptoms, are to be recorded on the eCRF. Each AE is to be evaluated for duration, intensity, and causal relationship with the study treatment or other factors.

All AEs occurring during the treatment period and/or occurring within 30 days of the last dose of Talabostat mesylate will be followed to the end of the study or until resolution. AEs will be graded according to the revised NCI CTCAE, Version 5.0, (see http://ctep.cancer.gov/reporting/ctc.html). AEs occurring 30 days after the last dose of Talabostat mesylate do not need to be reported unless the investigator considers the event to be related to Talabostat mesylate.

Investigators are required to report to the sponsor or sponsor's representative all AEs experienced during the active treatment period of the clinical trial and for 30 days following the last dose of Talabostat mesylate. All AEs that result in permanent discontinuation of study medication, whether serious or non-serious, must be reported to the sponsor.

Clinical laboratory data are to be collected in this study, and toxicity trends will be analyzed utilizing objective toxicity criteria.

The causality criteria of related and possibly related will be considered “related” to the study drug(s) for regulatory reporting requirements.

### Reporting Serious Adverse Events

Any SAE or death occurring during the treatment period and/or within 30 days following the last dose of study medication must be reported to the sponsor or sponsor's representative within 24 hours of first knowledge of the occurrence. If any SAE occurs, study treatment should be interrupted or discontinued at the discretion of the physician investigator.

### Adverse Event Follow-Up

Patients are to be monitored for AEs throughout the treatment period and for a minimum of 30 days after their last dose of Talabostat mesylate.

### Pharmacokinetic Assessments

Sparse pharmacokinetic sampling will be performed at the time points described in Appendix A for analysis of concentrations of Talabostat mesylate. Samples will be taken immediately before the last dose on Day 14 of Cycles 1, 2, and 3, and also up to 168 hours after the C3D14 dose (with that final sample taken just before the C4D1 dose). Pharmacokinetic data will be analyzed using a population pharmacokinetic approach.

### Pharmacodynamic Assessments

Whole blood samples will be collected at the time points described in Appendix for analysis of relevant immune effector cytokines and various immunological effector cells, including neutrophils, MDSCs, dendritic cells, CAF and T-cells.

### Disease Progression

Worsening signs and symptoms of prostate cancer should be considered by the investigator as disease assessments are being made. PD is being assessed as an efficacy outcome in this study and should not be reported as an AE. However, deaths considered solely due to PD occurring within 30 days of the last dose of Talabostat mesylate should be reported as an AE outcome with the AE term reported as “progressive disease”.

Removal of Patients from the Study

Patients may be discontinued from the study for any of the following reasons:
Investigator recommends discontinuation and documents the reason(s)
There is a need for any treatment not allowed by the protocol
Patient's decision to withdraw consent or discontinue for any reason
There is an unacceptable AE thought to be related to study medication
At the sponsor's request

Any patient who discontinues during the treatment period should return to complete safety and disease assessments (see Appendix A and Appendix B).

### Study Completion

The study will be considered complete when all patients have been followed to disease progression; are lost to follow-up, death, or withdrawal due to toxicity; patient's request; or investigator's discretion; and have completed all end-of-study treatment procedures.

### Study Medication

Study medication was administered in 21-day cycles.

### Talabostat Mesylate Dosage and Administration

Talabostat mesylate tablets contain valine-proline boronic acid formulated as the methanesulfonate salt. Current dosage strengths include 0.05 mg and 0.2 mg tablets for oral administration.

The starting dose regimen of Talabostat mesylate (i.e., the dose regimen in Cohort 1) was 0.4 mg QD on Days 1 to 14 every 21 days. The Talabostat mesylate dose regimen for any patient depends on the cohort in which the patient was enrolled in the Lead-in Stage. Additional dosing schedules (e.g., 0.6 mg QD) were also evaluated during the Lead-in Stage.

Talabostat mesylate was administered orally as 0.2 mg tablets. Patients will take 2 or 3 tablets daily and administered once a day (as 2 or 3 tablets), twice a day (as 1+1 or 1+2 tablets taken AM and PM) or thrice a day (as 1+1+1 tablets given at different times during the day), on days 1 to 14 of each cycle, for a total daily dose of 0.4 or 0.6 mg. Talabostat mesylate will be continued until disease progression or unacceptable toxicity.

On days when pharmacodynamic studies are being performed, Talabostat mesylate should be administered at the study center, and should be administered at (approximately) the same time of day on each treatment day in the cycle. In cycles in which pharmacodynamics are not evaluated, Talabostat mesylate also should be administered at (approximately) the same time of day on each treatment day in the cycle, preferably 0800 hours.

### Dose Adjustments of Talabostat Mesylate Secondary to Toxicity

Talabostat mesylate dose modifications within a treatment cycle are not permitted in Cycle 1 in the absence of DLT. In Cycle ≥2, dose modifications within a treatment cycle will be at the discretion of the investigator. Doses held because of AEs should not be made up on subsequent days within or following a cycle. A dose that is missed for reasons other than an AE (i.e., the patient forgets to take a dose) may be administered on days subsequent to scheduled doses; any such adjustments should be discussed with the Medical Monitor or designee. Under no circumstances should missed doses be made-up on a day when the patient is already taking a planned dose (i.e., no “doubling-up” to account for missed doses).

If an SAE thought to be related to Talabostat mesylate occurs during the treatment period, dosing of Talabostat mesylate should be interrupted in that patient until the SAE resolves. If the investigator wishes to continue the patient on Talabostat mesylate, the sponsor should be contacted to discuss continuing Talabostat mesylate at the same or reduced dose.

The most frequently observed AEs that appear to be characteristic of Talabostat mesylate are edema/peripheral swelling, hypotension, dizziness, and hypovolemia. These events, including edema, tend to be manageable and reversible and usually resolve following a drug hold. Talabostat mesylate should be held for Grade 2 or higher episodes of such events, until resolution of these AEs. Talabostat mesylate can be restarted at full dose after resolution of these AEs, including edema. For other Grade 2 or higher AEs deemed related to Talabostat mesylate, or for edema that has not responded to drug hold, the dose of Talabostat mesylate can be reduced by 0.2 mg decrements at the discretion of the investigator.

Discontinuation of Talabostat mesylate should occur for any life-threatening AE, or for Grade 2 or higher treatment-related AEs that do not respond to dose reduction to 0.2 mg. If Talabostat mesylate is discontinued due to an AE, all termination from treatment procedures and assessments must be performed.

Monitoring of Patient Compliance with Talabostat Mesylate Study Medication

All Talabostat mesylate dosing containers must be returned to the clinic at each visit. Patients should be queried regarding their compliance with the dosing regimen and medication containers should be reviewed at each visit to determine if any doses of Talabostat mesylate have been missed, and the number of missed doses recorded. Patients must be at least 75% compliant with taking Talabostat mesylate in Cycles 1 and 2 in order to be included in the per-protocol efficacy analyses.

### Talabostat Mesylate Description and Storage

Talabostat mesylate was supplied as 0.05 mg and 0.2-mg tablets in high-density polyethylene bottles with desiccant and child-resistant caps. 30 tablets were provided in each bottle. Supplies of Talabostat mesylate were appropriately labeled for clinical trial material. Talabostat mesylate should be stored under refrigerated conditions between 2° C. to 8° C. (33° F. to 46° F.).

### Dose Modifications of Pembrolizumab

Dose modification of Pembrolizumab should be in accordance with the current Package Insert for AEs deemed related to Pembrolizumab as discussed here.

Pembrolizumab should be withheld for any of the following:
Grade 2 pneumonitis (see US Package Insert—Warnings and Precautions Section 5.1)
Grade 2 or 3 colitis (see Warnings and Precautions [5.2])
Grade 3 or 4 endocrinopathies (see Warnings and Precautions [5.4])
Grade 2 nephritis (see Warnings and Precautions [5.5])
Grade 3 severe skin reactions or suspected Stevens-Johnson syndrome (SJS) or toxic epidermal necrolysis (TN) (see Warnings and Precautions [5.6])
AST or ALT >3 to 5×ULN or total bilirubin >1.5 to 3×ULN
Any other Grade 2 or 3 treatment-related adverse reaction, based on the severity and type of reaction (see Warnings and Precautions [5.7]).

Clinical investigators will be advised to resume PEMBROLIZUMAB in patients whose adverse reactions recover to Grade 0 or 1.

For AEs where causality is not evident, discussion of dose modification with the medical monitor is recommended.

### Discontinuation of Pembrolizumab

Dose modification, including discontinuation, of Pembrolizumab, should be in accordance with the current Package Insert.

Permanently discontinue Pembrolizumab for any of the following:
Any life-threatening adverse reaction (excluding endocrinopathies controlled with hormone replacement therapy)
Grade 3 or 4 pneumonitis or recurrent pneumonitis of Grade 2 severity (see Warnings and Precautions [5.1])
Grade 3 or 4 nephritis (see Warnings and Precautions [5.5])
Grade 4 severe skin reactions or confirmed SJS or TEN (see Warnings and Precautions [5.6])
AST or ALT >5×ULN or total bilirubin >3×ULN
For patients with liver metastasis who begin treatment with Grade 2 AST or ALT, if AST or ALT increases by ≥50% relative to baseline and lasts for at least 1 week
Grade 3 or 4 myocarditis, encephalitis, or Guillain-Barré syndrome (see Warnings and Precautions [5.7])
Grade 3 or 4 infusion-related reactions (see Warnings and Precautions [5.8])
Inability to reduce corticosteroid dose to 10 mg or less of prednisone or equivalent per day within 12 weeks
Persistent Grade 2 or 3 adverse reactions (excluding endocrinopathies controlled with hormone replacement therapy) that do not recover to Grade 0 or 1 within 12 weeks after last dose of Pembrolizumab.

### Data Analysis and Statistical Considerations

A Statistical Analysis Plan (SAP) will be written to address the analysis of data recorded in the clinical database as well as laboratory data, pharmacodynamic and other data transferred to Novella Clinical. The analysis of safety lead-in data to determine the Talabostat mesylate dose to use for the Efficacy Stage of the study will initially use data management listings. These lead-in data will be combined with data from the Efficacy Stage of the study and will be presented in the clinical study report.

Continuous variables, including baseline characteristics, will be summarized by reporting the number of observations, mean, standard deviation, median, minimum and maximum.

Categorical/discrete variables will be summarized using frequency tables showing the number and percentage of patients within a category. Time-to-event data will be summarized using the Kaplan-Meier method.

Unless indicated otherwise, summary statistics will be reported for observed data only. Missing data will not be imputed. If a baseline value is missing, no change from baseline will be calculated. Baseline is defined as the last available observation prior to the first administration of study drug on C1D1.

The handling of missing data will be specified in the SAP along with the methods used for reporting the endpoints.

Statistical analyses will be carried out by Novella Clinical using SAS Version 9.4 or higher. Any deviations from the SAP will be reported in the clinical study report.

### Analysis Populations

The intent-to-treat (ITT) analysis population will consist of patients who meet the eligibility criteria.

The response evaluable patient population will consist of patients who have completed at least 2 cycles of treatment with combined Talabostat mesylate and Pembrolizumab, with at least 1 post-baseline response assessment made by the investigator(s).

The safety population will consist of all patients who received any dose of Talabostat mesylate/Pembrolizumab, either during the Lead-in or Efficacy Stages of the study.

The pharmacodynamic analysis population will consist of all patients who received any dose of Talabostat mesylate/Pembrolizumab and have DPP activity or cytokine levels measured at least once.

### Analysis of Demographics and Baseline Characteristics

Demographics and baseline disease characteristics will be summarized and listed for the ITT analysis population. If the number of patients in the ITT analysis population differs substantively from the number of patients in the response evaluable or safety analysis population, demographics and baseline characteristics for these analysis populations may be presented.

### Efficacy Data Analysis

The primary and secondary efficacy parameters are defined herein. The objective response rate is defined as the number of patients with a CR or PR over all evaluable patients. Response will be determined by RECIST 1.1 Criteria. The duration of response is defined as the time interval measured in days between the first date when the criteria for objective response are met and the first date on which objective progression is documented. Patients who do not experience disease progression during the treatment and follow-up period, and who do not die during the treatment period will have their event time censored on the last study date that objective tumor assessments verified lack of disease progression. One day will be added to each calculation to account for the designation of the first day of treatment as Study Day 1. Patients who achieve a PR and then a CR will have times calculated using the date of the PR. Radiographic PFS is defined as the time from the date of initiation of protocol therapy to date measurement criteria are first met for PD by RECIST 1.1/PCWG3 criteria or death from any cause, whichever occurs first. Patients lacking an evaluation of tumor response will have their event time censored on Day 1. Patients not experiencing disease progression during the treatment and extended disease-assessment period, and who do not die during the treatment period will have their event time censored on the last date that objective tumor assessments verified lack of disease progression. A patient's data will be censored at the point he/she receives new cancer therapy in the absence of documented disease progression. One day will be added to each calculation to account for the designation of the first day of treatment as Study Day 1. Progression Free Survival (PFS) is defined as the time from the date of initiation of protocol therapy to date measurement criteria are first met for PSA progression by PCWG3 criteria. Patients who have not met definitive criteria for progression will be censored at the latest date of assessment. Overall Survival: Survival time is the difference in days between the date of death and the first date of study treatment (+1 day). Patients not expiring will have their survival times censored on the last date of known contact on which the patient was documented to be alive. Treated patients lacking data beyond the start of therapy will have their survival times censored on Day 1.

### Analysis of the Primary Efficacy Parameter

### Stage 1 Analysis

When 15 patients have completed approximately 6 cycles of treatment and have 2 post-baseline tumor assessments and PSA or CTC measurements, the number of patients who meet the composite response criteria of achieving 1 or more the following: 1) objective response by RECIST 1.1 criteria, 2) ≥50% decline from baseline in serum PSA by Week 12 of treatment, or 3) CTC conversion from >5/7.5 mL to <5/7.5 mL per Veridex assay by completion of Week 12 of protocol therapy will be evaluated.

Using minimax 2-stage Simon design, if 2 or fewer out of 15 stage 1 patients meet at least 1 of the composite response criteria, enrollment will be stopped. This will indicate that data to date are consistent with the null hypothesis that the composite response rate is 15% or less, thereby rejecting the alternative hypothesis that the composite response rate is 35%. If 3 or more out of 15 patients meet at least 1 of the composite response criteria, 13 more patients will be enrolled and treated to proceed to stage 2, for a total of 28 patients in both stages

### Stage 2 Analysis

When the additional 13 patients in stage 2 enrolled and treated have completed approximately 6 cycles of treatment, 2 post-baseline assessments of tumor, and PSA and CTCs measurements, the number of patients who meet at least 1 of the 3 criteria for the composite endpoint will be evaluated. If the total number of patients who meet the composite endpoint in 28 patients in both stages is 7 or less, then data are consistent with the null hypothesis of composite endpoint rate of 15% or lower with nominal 0.05 1-sided significance level. If the number of patients who meet the composite endpoint is 8 or more, then the data are consistent with the composite endpoint rate of at least 35%. The composite endpoint rate across 2 stages and its exact 95% confidence interval (CI) will be calculated as if data are collected in a single stage. This approach that ignores the sequential statistical testing may lead to biased point estimate of the composite endpoint rate and the CI may not provide the stated coverage probability, but is generally accepted when the event rate is relatively small.

### Sensitivity Analysis

The composite endpoint rate will also be calculated for the ITT analysis population. Patients in the ITT population with missing composite endpoint will be considered non-responders (e.g., not 1 of the 3 criteria for composite endpoint is met).

### Analysis of Secondary Parameter(s): Time-to-Event Response

The distribution of time-to-event response including rPFS, PSA, PFS, DOR, and OS will be estimated by Kaplan-Meier methodology. The medians of these time-to-event efficacy responses, if available, and their 2-sided 95% CI, will be reported. In addition, the proportions of patients with events at selected time points, together with their 2-sided 95% CI will be presented. The calculations will be performed based on fixed sample, single stage design.

The primary analysis will be performed using the ITT analysis population. As a supplement, time-to-event analysis will be performed using the response evaluable analysis population.

Analyses on duration of overall objective response will be performed for all ITT analysis population who achieve confirmed PR or CR. The number of CR and PR patients may be small, and thereby limit use of the Kaplan-Meier method to provide reliable information. In this case, descriptive statistics or listings will be provided.

After discontinuing the study medication, patients may be treated with additional therapy. Data collected after patients have been treated with additional therapy will not be used to evaluate the duration of objective response.

### Analysis of Secondary Endpoints: DPP Activity and Cytokine Levels

The analysis of the secondary efficacy endpoints including DPP activity and cytokine levels will be reported for the PD analysis population. The proportion of patients who exhibit DPP activity will be reported and descriptive statistics for levels of cytokines previously shown to be modulated by Talabostat mesylate in human will be reported.

### Analysis of Exploratory Endpoints

The exploratory endpoints will be analyzed using the response evaluable analysis population.

The proportion of patients who meet the iRECIST criteria will be presented.

At the minimum, the proportion of who experience clinical benefit (PR, CR) and OS will be reported.

Whenever feasible, cross tabulation of clinical outcomes by presence/absence of relevant immune effector cytokines and various immunological effector cells, including neutrophils, MDSCs, dendritic cells, CAF and T-cells in blood in tumor tissues will be presented.

The baseline PD-L1 tumor expression in metastatic tumor tissue and CTCs will be cross-tabulated with subsequent clinical outcomes. Similarly, the baseline and on-treatment circulating tumor neoantigens and T-cell repertoire, and baseline tumor mRNA immune profiling panel will be cross-tabulated with clinical outcomes with regards to response and safety.

### Analysis of Treatment Exposure

Descriptive summary statistics will be provided for total number of cycles, doses, average dose administered, and duration of treatment.

### Analysis of Patient Study Disposition

The number of patients enrolled in the ITT analysis population, number of patients in the safety, response evaluable, and pharmacodynamic populations will be reported.

The number of patients discontinuing the study over time will be summarized for the ITT population.

### Statistical Power and Sample Size Considerations

The sample size is calculated to reflect Simon's 2-stage design for the Efficacy Stage. In Simon's 2-stage design, an initial number of patients is enrolled and evaluated for stopping for futility; that is data are tested for consistency with the null hypothesis using a 1-sided test. If the analysis indicates that data are consistent with the null hypothesis, the study will be stopped for futility. Otherwise, the study proceeds to a second stage where additional number of patients will be enrolled to test if data support either the null or alternative hypothesis. The Simon 2-stage design only considers stopping for futility at stage 1, and admissible sample sizes that meet pre-specified power and type 1 error are considered for both study stages.

A total sample size of 28 patients, 15 patients at stage 1 and 13 patients at stage 2, will be treated with combined Talabostat mesylate and Pembrolizumab in order to detect with 80% power an alternative hypothesis percentage of patients who meet the composite endpoint of 35% versus the null hypothesis that the percentage of 15%, with early stopping for futility at stage 1, in a 1-sided test with 0.05 significance level (actual value is 0.0461). Two or fewer patients who meet the composite endpoint at stage 1 will trigger early stopping. Seven or fewer patients among 28 patients in both stages will lead to rejecting the alternative hypothesis that the composite endpoint rate is at least 35%.

### Safety Analyses

All patients in the safety population will be included in the final summaries and listings of safety data, separately for the lead-in patients grouped into 2 dose cohorts (0.4 and 0.6 mg). Summaries of AEs and other safety parameters will be provided as appropriate. Emphasis in the analysis of AEs will be placed on those that are treatment-emergent through 30 days after last dose of Talabostat mesylate/Pembrolizumab.

Frequencies of patients experiencing at least 1 AE will be displayed by body system and preferred term according to Medical Dictionary for Regulatory Activities (MedDRA) terminology. Detailed information collected for each AE will include: a description of the event, duration, whether the AE was serious, nature of the event (single episode versus multiple episode), intensity (i.e., NCI CTCAE grade), relationship to study drug, action taken, clinical outcome, and whether the AE resulted in surgery or alternate procedures. Intensity (severity) of the AEs will be graded according the NCI CTCAE. The latest version of MedDRA and NCI CTCAE will be used.

Summary tables will be prepared to show the number of patients reporting AEs, the frequency of patient reports, and corresponding percentages. Percentages will be calculated using the number of patients in the safety population as the denominator. Within each table, the AEs will be categorized by MedDRA body system and preferred term. Additional subcategories will be based on event intensity and relationship to study drug. AE data will be presented across all cycles and for each cycle. The denominator for each cycle is those patients available at the start of the cycle who received a dose of Talabostat mesylate for that cycle.

To the extent possible, AE relationship to either Talabostat mesylate or Pembrolizumab will be identified.

Individual patient listings will be prepared for all AE data.

ECG, vital signs, and ECOG performance status will be summarized by visits/cycles, using descriptive statistics applicable to continuous or categorical measures of these additional safety data. Summaries for the Lead-in and Efficacy Stages will be presented.

### Replacement of Patients

Patients who are assigned a patient number and who do not receive at least 2 cycles of Talabostat mesylate will be replaced.

### Results:

### Stage 1—Lead in Stage—Interim Data

Three patients were treated at the initial dose level for at least 4 cycles. All patients remain on active treatment. No DLT or SAEs were reported. Grade 3 treatment related adverse events (TRAE) were limited to thrombocytopenia requiring transfusion in 1 patient. The only TRAE reported in more than one patient was hypocalcemia (2 patients). Safety assessment of Talabostat mesylate+Pembrolizumab is ongoing at the final dose escalation cohort.

### Conclusions:

The combination of Talabostat mesylate (0.4 mg QD on days 1 to 14 of 21-day cycle) plus pembrolizumab (200 mg IV on day 1 every 21 days) is safe in patients with CRPC.

TABLE 5
Phase 1b - Safety Lead-in stage - Patient disease history

| | | No of subjects | |
|---|---|---|---|
| | | Talabostat | Talabostat |
| | | mesylate | mesylate |
| | | (0.4 mg) + | (0.6 mg) + |
| | | Pembrolizumab | Pembrolizumab |
| Disease History | | N = 3 | N = 2 |
| Most Recent | Adenocarcinoma | 3 | 1 |
| Histopathology | Adenocarcinoma | — | 1 |
| | with small-cell | | |
| | or | | |
| | neuroendocrine | | |
| | features | | |
| | Primary Small | — | — |
| | cell or | | |
| | neuroendocrine | | |
| | carcinoma | | |
| Prior Systemic | ADT therapy | 3 | 2 |
| Therapies | Chemotherapy | 3 | 1 |
| | Radiotherapy | 2 | 2 |
| Type of | PSA only | 1 | — |
| progression at | Bone +/− PSA | 1 | 1 |
| study entry | Bone + nodal | 1 | — |
| | disease +/− PSA | | |
| | Visceral +/− | — | 1 |
| | other | | |

TABLE 6
Phase 1b - Safety Lead-in stage - Safety data

| | No. of Patients |
|---|---|
| | Talabostat mesylate (0.4 mg) + |
| Safety | Pembrolizumab N = 3 |
| Any DLT | 0 |
| Any treatment related SAE | 0 |
| Any cause AE | 3 |
| Any Talabostat mesylate treatment | 2 |
| related AE* | |
| Any Pembrolizumab treatment | 2 |
| related AE* | |
| Any Talabostat mesylate or | 1** |
| Pembrolizumab treatment related | |
| Grade 3 or 4 AE | |
| Common treatment related AEs | 2 |
| (>1 Pt) | |
| Hypocalcemia | |

*Includes possibly-related events
**Grade 3 thrombocytopenia requiring transfusion

TABLE 7
Phase 1b- Safety Lead-in stage - Disease state

| | | Talabostat mesylate |
|---|---|---|
| | | (0.400 mg) + |
| | | Pembrolizumab |
| Composite responses | | N = 3 |
| RECIST Response | SD | 2 |
| after 3 cycles | Non-CR/Non-PD | 1* |
| (9 weeks) per | | |
| Investigator | | |

| | |
|---|---|
| CTC conversion from >5/7.5 | Data Not available |
| to <5/7.5 by Week 12 | |
| PSA decline >50% | 0 |
| by Week 12 | |

*Pt #3 with only bone mets at baseline

TABLE 8
Phase 1b- Safety Lead-in stage - Early PK observations
Plasma concentrations (ng/ml) of Talabostat mesylate in
the current study and healthy volunteer study CA168-002

| | Current study | | healthy volunteer |
|---|---|---|---|
| | 0.4 mg qd Day 14 (Cycle 3) | | study CA168-002 |
| Time Points | Sub101 Cycle 3 | Sub102 Cycle 2 | 300 mcg qd Day 7 |
| Pre-dose | 2.0 | 2.7 | 1.3 |
| 2 hours | 6.3 | | 4.8 |
| 6 hours | 4.2 | | 2.5 |

The Talabostat mesylate formulation herein is consistent with previously reported results from Point therapeutics (healthy volunteer study CA168-002) based on the initial plasma concentration data from 2 patients in the current study.

TABLE 9
Phase 1b- Safety Lead-in Stage - Subject disposition

| | | | | | |
|---|---|---|---|---|---|
| Patient # | C1D1 | C2D1 | C3D1 | C4D1 | C5D1 |

Cohort #1: Talabostat mesylate 0.4 mg + Pembrolizumab (N = 3)

| | | | | | |
|---|---|---|---|---|---|
| #1 | X | X | X | X | X |
| #2 | X | X | X | X | X |
| #3 | X | X | X | X | |

Cohort #2: Talabostat mesylate 0.6 mg + Pembrolizumab (N = 3)

| | |
|---|---|
| #4 | X |
| #5 | X |
| #6 | |

### Summary:

In the Phase 1b safety lead-in portion of this study in subjects with CRPC:
Safety of Talabostat mesylate+Pembrolizumab has been demonstrated in the initial cohort with no SAEs or DLTs and a low rate of treatment-related ≥Grade 3 events
Assessment of Talabostat mesylate+Pembrolizumab combination safety is ongoing in the final dose escalation cohort
Preliminary pharmacokinetics of Talabostat mesylate are within expectations based on prior data
All subjects remain on treatment

The Phase 2 portion of this study will be limited to subjects with SCNC, an aggressive form of prostate cancer, and will assess the anti-tumor activity of the combination of Talabostat mesylate+Pembrolizumab in a setting where checkpoint inhibitor monotherapies have demonstrated limited clinical benefit.

### Study Schedule of Assessments

TABLE 10

| | Screen | | | | | | | | | | | | | | | | |
|---|---|---|---|---|---|---|---|---|---|---|---|---|---|---|---|---|---|
| | Period | Cycle 1 | | | | | Cycle 2 | | | | | >Cycle 2 | | | | | |
| | Screen/Cycle Day (D): | | | | | | | | | | | | | | | | |
| | D-28 to | | | | | | | | | | | | | | | EOT | FU |
| | D-1 | D 1 | D 2 | D 8 | D 14 | D 15 | D 1c | D 2 | D 8 | D 14 | D 15 | D 1c | D 8 | D 14 | D 15 | Visit^{a} | Visit^{b} |
| Informed consent | X | | | | | | | | | | | | | | | | |
| Inclusion and | X | X | | | | | | | | | | | | | | | |
| exclusion criteria | | | | | | | | | | | | | | | | | |
| Demographics | X | | | | | | | | | | | | | | | | |
| Adverse Event | X | X | | X | | X | X | | X | | X | X | X | | X | X | X |
| Assessment | | | | | | | | | | | | | | | | | |
| Concomitant | X | X | | X | | X | X | | X | | X | X | X | | X | X | X |
| Medications | | | | | | | | | | | | | | | | | |
| Prostate cancer | X | | | | | | | | | | | | | | | | |
| treatment history | | | | | | | | | | | | | | | | | |
| Archival tumor | X | | | | | | | | | | | | | | | | |
| collection | | | | | | | | | | | | | | | | | |
| Central pathology | X | | | | | | | | | | | | | | | | |
| review of primary | | | | | | | | | | | | | | | | | |
| or metastatic | | | | | | | | | | | | | | | | | |
| prostate cancer | | | | | | | | | | | | | | | | | |
| tissue | | | | | | | | | | | | | | | | | |
| Imaging and Other | | | | | | | | | | | | | | | | | |
| Bone scintigraphy | X | | | | | | | | | | | X | | | | X | |
| CT/MRI | X | | | | | | | | | | | X | | | | X | |
| Tumor assessment | X | | | | | | | | | | | X | | | | X | |
| Study Drug | | | | | | | | | | | | | | | | | |
| Administration | | | | | | | | | | | | | | | | | |
| Talabostat | | Day | | Day | | Day | | | | | | | | | | | |
| mesylate | | 1-14 | | 1-14 | | 1-14 | | | | | | | | | | | |
| administration | | | | | | | | | | | | | | | | | |
| Pembrolizumab | | X | | | | | X | | | | | X | | | | | |
| administration | | | | | | | | | | | | | | | | | |
| Clinical | | | | | | | | | | | | | | | | | |
| Procedures | | | | | | | | | | | | | | | | | |
| Physical | X | X | | X | | X | X | | X | | X | X | | | | X | |
| examination | | | | | | | | | | | | | | | | | |
| Medical history/ | X | | | | | | | | | | | | | | | | |
| current medical | | | | | | | | | | | | | | | | | |
| conditions | | | | | | | | | | | | | | | | | |
| ECOG performance | X | X | | X | | X | X | | X | | X | X | | | | X | |
| status | | | | | | | | | | | | | | | | | |
| ECOG performance | X | X | | X | | X | X | | X | | X | X | | | | X | |
| status | | | | | | | | | | | | | | | | | |
| Vital signs (sitting | X | X | | X | | X | X | | | | X | X | | | | | |
| and standing BP, HR, | | | | | | | | | | | | | | | | | |
| body temperature, RR) | | | | | | | | | | | | | | | | | |
| Height | X | | | | | | | | | | | | | | | | |
| Weight | X | X | | | | | X | | | | | X | | | | X | |
| Metastatic tumor | X | | | | | | | | | | | | | | | | |
| biopsy | | | | | | | | | | | | | | | | | |
| ECG | X | X | | | | | X | | | | | X | | | | X | |
| Clinical | | | | | | | | | | | | | | | | | |
| Laboratory Tests | | | | | | | | | | | | | | | | | |
| Hematology | X | X | | X | | X | X | | X | | X | X | X | | X | X | |
| (CBC plus | | | | | | | | | | | | | | | | | |
| differential | | | | | | | | | | | | | | | | | |
| [5-part or | | | | | | | | | | | | | | | | | |
| auto-analyzer]) | | | | | | | | | | | | | | | | | |
| Serum chemistry | X | X | | X | | X | X | | X | | X | X | X | | X | X | |
| Liver function | X | X | | X | | X | X | | X | | X | X | X | | X | X | |
| tests | | | | | | | | | | | | | | | | | |
| Serum PSA | X | X | | | | | X | | | | | X | | | | | |
| Urinalysis | X | X | | | | | X | | | | | X | | | | X | |
| Whole blood | | X | X | | X | X | X | X | | X | X | | | | | | |
| collection for | | | | | | | | | | | | | | | | | |
| immune parameters | | | | | | | | | | | | | | | | | |
| Enumeration | | X | | | | | X | | | | | X | | | | X | |
| of CTCs by | | | | | | | | | | | | | | | | | |
| Veridex assay | | | | | | | | | | | | | | | | | |
| Pharmacokinetic | | | | | Xk | | | | | Xk | | | | Xl | Xl | | |
| blood sampling | | | | | | | | | | | | | | | | | |

Abbreviations: BP = blood pressure; CxDx = Cycle (number) Day (number); CBC = complete blood count; CT = computed tomography; CTC = circulating tumor cell; ECG = electrocardiogram; ECOG = Eastern Cooperative Oncology Group; EOT = End of Treatment; FU = follow-up; HR = heart rate; MRI = magnetic resonance imaging; PSA = prostate-specific antigen; RR = respiratory rate.

### Study Follow-Up

a. After discontinuation of study drugs, patients will complete an EOT visit within 21 days after the last study drug dose.
b. A Safety follow-up visit is to be conducted 30 days (±7 days) after the last dose of study drug and later if drug-related AEs have not resolved at that time. Thereafter, patients without documented disease progression (PD) will be followed every 90 days for disease assessments until documentation of PD. After documentation of PD, patients will be followed every 90 days for survival status; such follow-up will likely be conducted by telephone.
c. Day 1 of Cycle 2 and all subsequent cycles will be 21 days (±3 days) after the previous dose of study drug was administered.
d. Tumor assessment must include cross-sectional imaging (MRI or CT scanning with intravenous contrast whenever possible) of the chest/abdomen/pelvis plus whole-body bone scan. Other body sites (e.g., neck) to be included as clinically indicated. Tumor assessment to be performed at Screening, C4D1 (±7 days), C7D1 (±7 days), C10D1 (±7 days), and Day 1 (±7 days) of every 3rd cycle thereafter.
e. Tumor biopsy is optional in the Lead-in Stage, and mandatory in the Efficacy Stage. Requirement may be waived if no safely accessible lesion OR patient has available archival metastatic tumor tissue.
f. ECGs should be performed in triplicate, prior to collection of blood samples. At Screening only, QT interval corrected for heart rate using Bazett's formula (QTcB) will be measured.
g. Serum chemistry to include: sodium (Na), potassium (K), chloride (Cl), bicarbonate, calcium (Ca), magnesium (Mg), phosphate, blood urea nitrogen (BUN)/creatinine (Cr), and lactate dehydrogenase (LDH).
h. Liver function tests include aspartate aminotransferase, alanine aminotransferase, alkaline phosphatase, total bilirubin, albumin.
i. Whole blood for immune parameters will be collected on predose and 6 and 24 hours post-dose on C1D1, C1D14, C2D1, and C2D14. The Day 1, 24 hours samples will be collected prior to the Day 2 dose.
j. CTC enumeration will be performed on C1D1, C2D1 and C4D1 and then on D1 of every third cycle thereafter, and EOT visit.
k. Sample collected immediately before last dose is administered on Day 14 (patient diary to be kept to record the number of doses the patient has taken in the cycle).
l. Cycle 3 only; samples collected immediately before the last dose is administered on Day 14 and at 2, 6, 12, 24, 72, 120, and 168 hours after the last dose, with the 168-hour sample collected immediately prior to the C4D1 dose (patient diary to be kept to record the number of doses the patient has taken in the cycle).

### Detailed Study Procedures by Visit Screening Period (Day −28 to Day −1)

Informed consent will be obtained before any procedures are completed.

All clinical laboratories will be analyzed by the institutional or other local laboratory.

Within 28 days prior to the first planned dose of Talabostat mesylate, the patient should have the following assessments performed:
Informed consent
Review of inclusion and exclusion criteria to determine of the patient is eligible to participate in the study
Collection of demographic data
Assess for AEs
Documentation of all concomitant medications
Prostate cancer treatment history
Archival tumor collection
Central pathology review of primary or metastatic prostate cancer tissue
Tumor assessment, which must include cross-sectional imaging (MRI or CT scanning with IV contrast whenever possible) of the chest/abdomen/pelvis plus whole-body bone scan. Other body sites (e.g., neck) to be included as clinically indicated.
Metastatic tumor biopsy (optional for Lead-in Stage). This may be waived if in the absence of a safely accessible lesion OR if patient has available archival metastatic tumor tissue.
Physical examination (including vital signs [sitting and standing blood pressure, heart rate, respiratory rate and temperature] and physical measurements [height, weight, and review of systems])
Complete medical history
ECOG performance status
Obtain 12-lead ECG (including assessment of QTcB)
Clinical laboratory assessments:
Hematology (complete blood count [CBC] plus differential count)
Serum chemistry (sodium [Na], potassium [K], chloride [Cl], bicarbonate, calcium [Ca], magnesium [Mg], phosphate, blood urea nitrogen [BUN]/creatinine [Cr], and lactate dehydrogenase [LDH]).
Liver function tests (AST, ALT, alkaline phosphatase, total bilirubin, albumin)
Serum PSA
Urinalysis
Once a patient is deemed eligible for the study, assign sequential patient numbers at each site

### Assessments During the Treatment Period

### Cycle 1, Day 1

The following tests and procedures will be performed:
Review of inclusion and exclusion criteria to confirm that patient remains eligible to continue in the study
Assess for AEs
Query for concomitant medications
Dispense Talabostat mesylate for administration on Days 1-14, Cycle 1
Administer Pembrolizumab
Physical examination (including vital signs [sitting and standing blood pressure, heart rate, respiratory rate and temperature] and weight)
ECOG performance status
Obtain 12-lead ECG
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
Serum PSA
Urinalysis
Whole blood for immune parameters (predose, and 6 and 24 hours postdose, with the 24 hours post-dose sample collected prior to dosing on Day 2)
Blood sample collection in CellSave preservative tube for CTC enumeration

### Cycle 1, Day 8

The following tests and procedures will be performed:
Assess for AEs
Query for concomitant medications
Physical examination (including vital signs [blood pressure, heart rate, respiratory rate, and temperature])
ECOG performance status
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)

### Cycle 1, Day 14

The following tests and procedures will be performed:
Whole blood for immune parameters (pre-dose, and 6 and 24 hours post-dose, with the 24 hours post-dose sample collected prior to dosing on Day 15)
Collection of blood sample for pharmacokinetic analysis immediately before the final dose

### Cycle 1, Day 15

The following tests and procedures will be performed:
Assess for AEs
Query for concomitant medications
Physical examination (including vital signs [blood pressure, heart rate, respiratory rate, and temperature])
ECOG performance status
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)

### Cycle 2, Day 1

The following tests and procedures will be performed:
Assess for AEs
Query for concomitant medications
Dispense Talabostat mesylate for administration on Days 1-14, Cycle 2
Administer Pembrolizumab
Physical examination (including vital signs [blood pressure, heart rate, respiratory rate and temperature] and weight)
ECOG performance status
Obtain 12-lead ECG
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
Serum PSA
Urinalysis
Whole blood for immune parameters (pre-dose, and 6 and 24 hours post-dose, with the 24 hours post-dose sample collected prior to dosing on Day 2)
Blood sample collection in CellSave preservative tube for CTC enumeration

### Cycle 2, Day 8

The following tests and procedures will be performed:
Assess for AEs
Query for concomitant medications
Physical examination
ECOG performance status
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)

### Cycle 2, Day 14

The following tests and procedures will be performed:
Whole blood for immune parameters (pre-dose, and 6 and 24 hours post-dose, with the 24 hours post-dose sample collected prior to dosing on Day 15)
Collection of blood sample for pharmacokinetic analysis immediately before the final dose

### Cycle 2, Day 15

The following tests and procedures will be performed:
Assess for AEs
Query for concomitant medications
Physical examination (including vital signs [blood pressure, heart rate, respiratory rate, and temperature])
ECOG performance status
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
   Cycle 3 and subsequent cycles, Day 1

The following tests and procedures will be performed:
Assess for AEs
Query for concomitant medications
Dispense Talabostat mesylate for administration on Days 1-14 of that cycle
Administer Pembrolizumab
Physical examination (including vital signs [blood pressure, heart rate, respiratory rate and temperature] and weight)
ECOG performance status
Obtain 12-lead ECG
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
Serum PSA
Urinalysis
Tumor assessment, which must include cross-sectional imaging (MRI or CT scanning with intravenous contrast whenever possible) of the chest/abdomen/pelvis plus whole-body bone scan. Other body sites (e.g., neck) to be included as clinically indicated. Tumor assessment to be performed at C4D1 (±7 days), C7D1 (±7 days), C10D1 (±7 days), and Day 1 (±7 days) of every third cycle thereafter.
Blood sample collection in CellSave preservative tubes for CTC enumeration to be performed at C4D1 and the first day of every third cycle thereafter.
   Cycle 3 and subsequent cycles, Day 8 and Day 15

The following tests and procedures will be performed:
Assess for AEs
Query for concomitant medications
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
   Cycle 3 only, Days 14 to 21

The following tests and procedures will be performed:
Collection of blood samples for pharmacokinetic analysis immediately before the final dose on Day 14 and at 2, 6, 12, 24 (Day 15), 72 (Day 17), 120 (Day 19), and 168 (Day 21) hours following the Day 14 dose.
   End of Treatment Visit (within 21 Days after the Last Study Drug Dose)

The following tests and procedures will be performed:
Assess for AEs
Query for concomitant medications
Physical examination, including weight
ECOG performance status
Obtain 12-lead ECG
Clinical laboratory assessments:
Hematology (CBC plus differential count)
Serum chemistry (Na, K, Cl, bicarbonate, Ca, Mg, phosphate, BUN/Cr, and LDH).
Liver function tests (AST, ALT, alkaline phosphatase, total bilirubin, albumin)
Urinalysis
Tumor assessment which must include cross-sectional imaging (MRI or CT scanning with intravenous contrast whenever possible) of the chest/abdomen/pelvis plus whole-body bone scan. Other body sites (e.g., neck) to be included as clinically indicated.
Blood sample collection in CellSave preservative tube for CTC enumeration
Follow-up Visit (conducted 30 days [±7 days] after the last dose of study drug and later if drug-related AEs have not resolved at that time)

The following tests and procedures will be performed:
Assess for AEs
Query for concomitant medications

### Example 2

This is a Phase 2 Basket Study of Talabostat mesylate, a Small Molecule Inhibitor of Dipeptidyl Peptidases (DPP), administered in combination with Pembrolizumab in Patients with Advanced Solid Cancers.

### Study Objective(s)

Primary Objectives

The primary objectives of the study are:
To evaluate response rate per Response Evaluation Criteria in Solid Tumors (RECIST) and immune iRECIST in patients treated in cohort A and in patients treated in cohort B.
To evaluate dose-limiting toxicities (DLT) in the first 6 patients enrolled to the study.

Secondary Objectives

The secondary objectives of the study for cohort A and cohort B include:
To evaluate progression-free survival (PFS)
To evaluate duration of response (DOR)
To evaluate overall survival (OS)
To evaluate overall safety and tolerability

Exploratory Objectives
To evaluate the quantitative and qualitative effects of Talabostat mesylate in combination with pembrolizumab on relevant immune effector cytokines in blood
To evaluate the quantitative and qualitative effects of Talabostat mesylate in combination with pembrolizumab on various immunological effector cells, including neutrophils, myeloid derived suppressor cells (MDSCs), dendritic cells, cancer associated fibroblast (CAF), T-cells and macrophage density in pre-dose tumor biopsies and when feasible in post-dose tumor tissues.
To explore the predictive value of baseline PD-L1 tumor expression and tumor mutation burden (TMB) with clinical outcomes
To evaluate changes in serially collected blood circulating tumor DNA (ctDNA) to assess for tumor response and clonal evolution
To evaluate pre- and post-treatment PD-L1 PET/CT as a predictive tool for therapeutic efficacy.

### Study Design

This is an open-label, single-institution, Phase 2 study to determine the response rate of Talabostat mesylate administered orally and daily, combined with pembrolizumab, in patients with advanced solid cancers. The study will also assess other efficacy parameters, such as PFS, OS and DOR, as well as the safety the combined treatment. Bayesian optimal phase 2 (BOP2) design will be adopted to monitor efficacy. The study will consist of 2 stages:

1) Lead-in Stage (first 6 patients enrolled)—in which the safety and tolerability of the combination of Talabostat mesylate administered orally daily on Days 1 to 14 of a 21-day cycle plus pembrolizumab 200 mg administered intravenously (IV) on Day 1 every 21 days will be assessed and confirmed in patients with advanced solid cancers. The dose of Talabostat mesylate will be 0.6 mg.
2) Efficacy Stage (BOP2-Stage)—in which patients with advanced solid cancers will be treated with Talabostat mesylate combined with pembrolizumab. Patients enrolled to the Lead-in Stage will also be evaluated in the efficacy stage.

During the Lead-in Stage, patients will be observed for dose-limiting toxicity (DLT) during Cycle 1. Six patients will be treated initially with 0.6 mg Talabostat mesylate daily (Days 1 to 14) plus pembrolizumab 200 mg:
If >1 of the 6 original patients has a DLT in Cycle 1, the dose will be considered above the maximum tolerated dose (MTD) and additional 6 patients will be treated at the 0.4 mg Talabostat mesylate daily Days 1 to 14 dose level.
If ≤1 of the patients experience a DLT, the Efficacy Stage can commence
If >1 of the patients experience a DLT, a discussion will be held between the investigators and supporters as to how to proceed

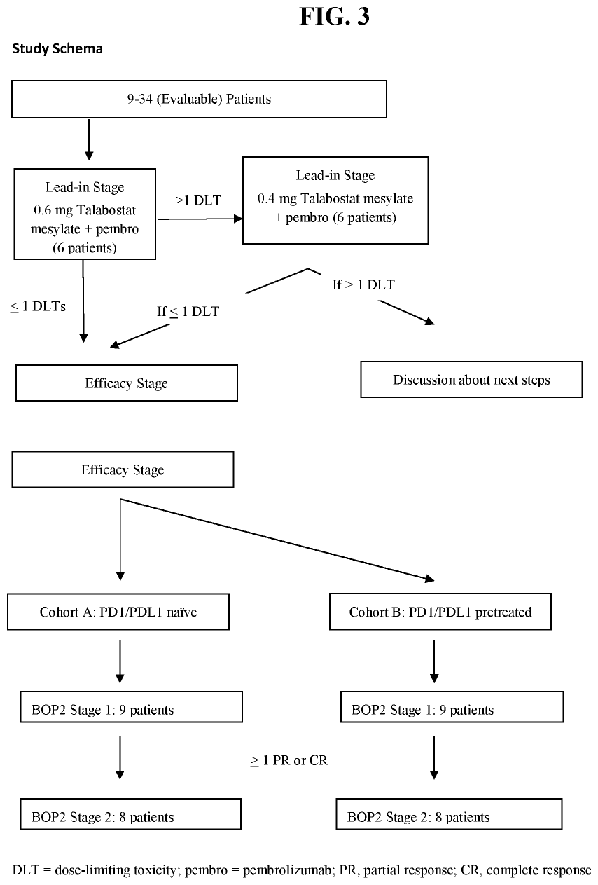

The study schema is presented in FIG. 3.

All safety data from all patients, who received at least one dose of study drug will be included in safety analysis. Unless doses were held because of DLT, a patient must have received >70% of their Talabostat mesylate in Cycle 1 (i.e., ≥30 of 42 planned doses) with pembrolizumab dosed on Day 1 of Cycle 1 to be eligible for DLT assessment.

Toxicities will be assessed by the investigator using the National Cancer Institute Common Terminology Criteria for Adverse Events (NCI CTCAE), version 5. The relationship of an AE to combination therapy (i.e., attribution to Talabostat mesylate and/or pembrolizumab) is to be assessed by the investigator using the criteria in the protocol.

A DLT is defined as any of the following AEs occurring during Cycle 1, regardless of investigator attribution to study treatment, unless the AE can be clearly and incontrovertibly attributed to an extraneous cause (e.g., disease progression) by the Principal Investigator:
Any Grade 4 laboratory abnormality, regardless of duration
Any Grade 3 non-hematologic AE, with the exceptions of Grade 3 nausea, vomiting, diarrhea, constipation, fever, fatigue, skin rash, or non-clinically significant laboratory abnormality that resolves to Grade ≤2 within 72 hours with optimal medical management.
Grade 3 thrombocytopenia with Grade >1 bleeding or requirement for platelet transfusion.
Grade 3 febrile neutropenia.
Grade 3 fever.
Grade 3 skin rash.
Laboratory abnormalities meeting Hy's law criteria (aspartate aminotransferase [AST] or alanine aminotransferase [ALT] >3× upper limit of normal [ULN] with concomitant total bilirubin >2×ULN).
Grade 3 transaminase (AST/ALT) elevation.
Any toxicity resulting in ≥30% held/skipped doses of Talabostat mesylate during Cycle 1.
Delay of Cycle 2 by ≥14 days due to toxicity.
Any other significant toxicity considered by the investigator and supporter's medical representatives to be dose-limiting.

### Efficacy Stage:

After assessment of the safety and confirmation of the Talabostat mesylate/pembrolizumab dose schedule to be used in the subsequent stage, the Efficacy Stage will begin. Eligible patients will receive oral Talabostat mesylate daily on Days 1 to 14 of a 21-day cycle plus pembrolizumab 200 mg administered IV on Day 1 every 21 days.

### Study Design Features (Both Stages):

In both Lead-in and Efficacy Stages, patients will be screened for study eligibility within 28 days before the first study drug dose after provision of written informed consent. Patients who are determined to be eligible, based on Screening assessments, will be enrolled in the study on Cycle (C)1, Day (D)1 (Baseline, before the first dose of Talabostat mesylate).

During treatment, patients will attend study center visits and have study evaluations performed as detailed in the Schedule of Assessments (Appendix A). All study visits will be conducted on an outpatient basis but may be conducted on an inpatient basis per the investigator's judgement.

All patients must have pre-treatment (prior to study treatment dosing) imaging (computed tomography [CT] scan of chest/abdomen/pelvis or magnetic resonance imaging [MRI] for baseline tumor measurements, as well as bone scintigraphy [BS]). Patients with skin, subcutaneous or lymph node metastases may also have tumor evaluations (including measurements, with a ruler) by means of physical examination. Patients with a history of central nervous system (CNS) malignant involvement or CNS symptoms should have either CT or MRI imaging of the brain performed to assess active CNS malignancy.

Tumor measurements and disease response assessments (CT or MRI; BS) are also to be performed at the end of Cycle 3 (approximately 9 weeks after the first study treatment dose), and then approximately every 9 weeks thereafter until development of progressive disease (PD). For patients with evidence of disease control (stable disease or better) at Week 27, tumor measurements and disease response assessments may be performed less frequently (approximately every 12 weeks) thereafter. Tumor measurements and disease response assessments also are to be performed at the End of Treatment (EOT) visit.

Study procedures are listed in Appendix A.

### Study Population

Approximately 6 to 12 and 24 to 48 patients who fulfill the eligibility criteria of the protocol will be enrolled during Lead-in and Efficacy Stages of the protocol, respectively. Patients enrolled to the Lead-in Stage will be evaluated and used for the efficacy stage.

### Eligibility Criteria

All patients must satisfy the following inclusion and exclusion criteria to be eligible for entry into the trial.

### Inclusion Criteria

1. Patient with a histology or cytology proven solid advanced cancer, which failed or is intolerant of standard therapies known to offer survival benefit unless standard therapies include PD1 or PD-L1 antibodies.
a. Lead-in stage: patient with advanced cancers meeting the criteria above with or without prior treatment with PD1/PDL1 antibodies. Patients with prior treatment with PD1/PDL1 antibodies should be relapsed.
b. Efficacy stage cohort A: patients with advanced cancers not previously treated with PD1/PDL1 antibodies.
c. Efficacy stage cohort B: patients with advanced cancers which have relapsed or progressed with PD1/PDL1 antibodies
2. Patient with a life expectancy of more than 3 months, in the opinion of the investigator.
3. Patient has Eastern Cooperative Oncology Group (ECOG) performance status of 0-2.
4. Patient is ≥12 years of age. Patients <18 years of age have to weigh ≥40 kgs.
5. Patients must have measurable disease by RECIST 1.1 and iRECIST. Disease amenable to a biopsy is not mandatory.
6. Patient's acute toxic effects of previous anticancer therapy have resolved to ≤Grade 1 except for Grade 2 peripheral neuropathy or any grade of alopecia.
7. Patient has adequate baseline organ function, as demonstrated by the following:
a. Serum creatinine ≤1.5 times institutional upper limit of normal (ULN) or calculated creatinine clearance >40 mL/min;
b. Serum albumin ≥2.5 g/dL;
c. Total bilirubin ≤1.5×ULN (for patients with known Gilbert syndrome ≤3×ULN);
d. Aspartate aminotransferase (AST) and alanine aminotransferase (ALT) ≤3× institutional ULN (patients with hepatic metastases must have AST/ALT ≤5×ULN).
8. Patient has adequate baseline hematologic function, as demonstrated by the following:
a. Absolute neutrophil count (ANC) ≥1.0×10⁹/L.
b. Hemoglobin ≥8 g/dL and no red blood cell transfusions during the prior 7 days.
c. Platelet count ≥75×10⁹/L.
9. Women of childbearing potential must have a negative serum or urine pregnancy test (minimum sensitivity 25 IU/L or equivalent units of human chorionic gonadotropin [HCG]) within 14 days prior to the initiation of treatment and/or postmenopausal women must be amenorrheic for at least 12 months to be considered of non-childbearing potential. Women of childbearing potential must agree and commit to the use of 2 highly effective methods of birth control throughout the duration of the study until at least 4 months following the last dose of study drug. Acceptable methods are defined as those that result, alone or in combination, in a low failure rate (ie, less than 1% per year) when used consistently and correctly, such as surgical sterilization, an intrauterine device, or hormonal contraception in combination with a barrier method. It is currently unknown whether Talabostat mesylate or pembrolizumab may reduce the effectiveness of systemically acting hormonal contraceptives; therefore, women using systemically acting hormonal contraceptives should add a barrier method. In certain countries (if permitted by law), WOCBP may agree to abide by heterosexual sexual abstinence during the time of participation in this study.
10. Male patients and their female partners of childbearing potential must agree and commit to use a barrier contraception (eg, condom with spermicidal foam/gel/film/cream/suppository) throughout the duration of the study until at least 60 days following the last dose of study drug, in addition to their female partners using either an intrauterine device or hormonal contraception and continuing until at least 4 months following the last dose of study drug. This criterion may be waived for male patients who have had a vasectomy >6 months before signing the ICF.
11. Patient has signed informed consent prior to initiation of any study-specific procedures or treatment.
12. Patient is able to adhere to the study visit schedule and other protocol requirements.

### Exclusion Criteria

1. Patient cannot swallow oral medication.
2. Patient has active central nervous system (CNS) metastases not controlled by prior surgery or radiotherapy (patient must be off steroids). Patients with signs or symptoms suggestive of brain metastasis are not eligible unless brain metastases are ruled out by brain MRI/CT.
3. Patient has received external-beam radiation or another systemic anticancer therapy within 14 days or 5 half-lives, whichever is shorter, prior to study treatment.
4. Patient has received treatment with an investigational systemic anticancer agent within 14 days prior to study drug administration.
5. Patient has an additional active malignancy that may confound the assessment of the study endpoints. Patients with the following concomitant neoplastic diagnoses are eligible: non-melanoma skin cancer and carcinoma in situ (including transitional cell carcinoma, anal carcinoma, and melanoma in situ). Patients with simultaneous cancers, which are not active and do not require treatment may be eligible contingent on discussion with the PI and supporter.
6. Patient has clinically significant cardiovascular disease (e.g., uncontrolled or any New York Heart Association Class 3 or 4 congestive heart failure, uncontrolled angina, history of myocardial infarction, unstable angina or stroke within 6 months prior to study entry, uncontrolled hypertension or clinically significant arrhythmias not controlled by medication).
7. Patient has a diagnosis of immunodeficiency or is receiving systemic steroid therapy at a prednisone equivalent dose of >10 mg daily for at least 1 week or other form of immunosuppressive therapy within 7 days prior to Cycle 1 Day 1 (C1D1).
8. Patient has uncontrolled intercurrent illness including, but not limited to, uncontrolled infection, disseminated intravascular coagulation, or psychiatric illness/social situations that would limit compliance with study requirements.
9. Patient has known positive status for human immunodeficiency virus active or chronic Hepatitis B or Hepatitis C. Patients with history of hepatitis B or C and undetectable viral load are eligible. Screening is not required.
10. Has a clinically significant upper gastrointestinal obstruction, abnormal physiological function or malabsorption syndrome that may affect the absorption of the study medication.
11. Patient has any medical condition which, in the opinion of the investigator, places the patient at an unacceptably high risk for toxicity.
12. Patient is pregnant or breast-feeding

### Study Methodology

### Concomitant Medications

### Permitted Medications/Therapies

The use of growth factors (e.g., granulocyte-colony stimulating factor [G-CSF]) is allowed as clinically indicated for the treatment of Grade ≥3 cytopenias.

Suggested supportive care measures for the management of AEs with potential immunologic etiology are outlined below. Where appropriate, these guidelines include the use of oral or IV treatment with corticosteroids as well as additional anti-inflammatory agents if symptoms do not improve with administration of corticosteroids. Note that several courses of steroid tapering may be necessary as symptoms may worsen when the steroid dose is decreased. For each AE, attempts should be made to rule out other causes such as metastatic disease or bacterial or viral infection, which might require additional supportive care.

### Prohibited Medications/Therapies

Enrolled patients may not receive investigational or approved anticancer agents including cytotoxic chemotherapy agents, anticancer tyrosine kinase inhibitors, or therapeutic monoclonal antibodies.

Palliative radiation is not permitted during study enrollment unless it is being performed for an existing, nonprogressive metastasis/symptoms and involves a narrow radiation port (e.g., solitary bone lesions).

Preclinical studies have demonstrated a low potential for Talabostat mesylate to inhibit the following major human liver CYP isoenzymes: CYP1A2, CYP2C8, CYP2C9, CYP2C19, CYP2D6, and CYP3A4. Further, relevant concentrations of Talabostat mesylate did not induce CYP3A4 or CYP1A2. Therefore, there are no prohibited medications based on CYP isoenzymes.

### Efficacy Assessments

Efficacy will be assessed during treatment using the RECIST 1.1 and iRECIST every 9 weeks (every 3 cycles). Details on RECIST and iRECIST are described in Appendix B and C.

### Safety Assessments

### Non-Serious Adverse Events

Investigators should assess for AEs at each visit. All AEs, including observed or volunteered problems, complaints, or symptoms, are to be recorded on the eCRF. Each AE is to be evaluated for duration, intensity, and causal relationship with the study treatment or other factors. MOCLIA will be used for eCRF in this study. Department (Investigational Cancer Therapeutics) database team will create the CRF based on protocol requirement and IND monitor will review and approve it once ready.

The investigator is responsible for monitoring the safety of patients who have entered the study. All AEs occurring during the treatment period and/or occurring within 30 days of the last dose of Talabostat mesylate and or pembrolizumab (investigational products, IPs) will be followed to the end of the study or until resolution. AEs will be graded according to the revised NCI CTCAE, Version 5.0, (see http://ctep.cancer.gov/reporting/ctc.html). AEs occurring 30 days after the last dose of IPs do not need to be reported unless the investigator considers the event to be related to IPs.

Recommended Adverse Event Recording Guidelines

| Attribution | Grade 1 | Grade 2 | Grade 3 | Grade 4 | Grade 5 |
|---|---|---|---|---|---|
| Unrelated | Phase I | Phase I | Phase I | Phase I | Phase I |
| | | | Phase II | Phase II | Phase II |
| | | | | Phase III | Phase III |
| Unlikely | Phase I | Phase I | Phase I | Phase I | Phase I |
| | | | Phase II | Phase II | Phase II |
| | | | | Phase III | Phase III |
| Possible | Phase I | Phase I | Phase I | Phase I | Phase I |
| | Phase II | Phase II | Phase II | Phase II | Phase II |
| | | Phase III | Phase III | Phase III | Phase III |
| Probable | Phase I | Phase I | Phase I | Phase I | Phase I |
| | Phase II | Phase II | Phase II | Phase II | Phase II |
| | | Phase III | Phase III | Phase III | Phase III |
| Definitive | Phase I | Phase I | Phase I | Phase I | Phase I |
| | Phase II | Phase II | Phase II | Phase II | Phase II |
| | | Phase III | Phase III | Phase III | Phase III |

### Reporting Serious Adverse Events

An adverse event or suspected adverse reaction is considered “serious” if, in the view of the investigator, it results in any of the following outcomes:
Death
A life-threatening adverse drug experience—any adverse experience that places the patient, in the view of the initial reporter, at immediate risk of death from the adverse experience as it occurred. It does not include an adverse experience that, had it occurred in a more severe form, might have caused death.
Inpatient hospitalization or prolongation of existing hospitalization
A persistent or significant incapacity or substantial disruption of the ability to conduct normal life functions.
A congenital anomaly/birth defect.

Important medical events that may not result in death, be life-threatening, or require hospitalization may be considered a serious adverse drug experience when, based upon appropriate medical judgment, they may jeopardize the patient or subject and may require medical or surgical intervention to prevent one of the outcomes listed in this definition.

Important medical events as defined above, may also be considered serious adverse events. Any important medical event can and should be reported as an SAE if deemed appropriate by the Principal Investigator or the IND supporter, IND Office.

All events occurring during the conduct of a protocol and meeting the definition of a SAE must be reported to the IRB in accordance with the timeframes and procedures outlined in “The University of Texas M. D. Anderson Cancer Center Institutional Review Board Policy for Investigators on Reporting Serious Unanticipated Adverse Events for Drugs and Devices”. Unless stated otherwise in the protocol, all SAEs, expected or unexpected, must be reported to the IND Office, regardless of attribution (within 5 working days of knowledge of the event).

All life-threatening or fatal events, that are unexpected, and related to the study drug, must have a written report submitted within 24 hours (next working day) of knowledge of the event to the Safety Project Manager in the IND Office.

Unless otherwise noted, the electronic SAE application (eSAE) will be utilized for safety reporting to the IND Office and MDACC IRB.

Serious adverse events will be captured from the time of the first protocol-specific intervention, until 90 days after the last dose of drug or earlier if the participant withdraws consent or starts a new anti-cancer therapy. Serious adverse events must be followed until clinical recovery is complete and laboratory tests have returned to baseline, progression of the event has stabilized, or there has been acceptable resolution of the event.

Additionally, any serious adverse events that occur after the 90-day time period that are related to the study treatment must be reported to the IND Office. This may include the development of a secondary malignancy.

### Adverse Event Follow-Up

Patients are to be monitored for AEs throughout the treatment period and for a minimum of 30 days after their last dose of Talabostat mesylate.

No further reporting of new AEs is required after the initiation of any subsequent chemotherapy or more than 30 days following the last dose of study medication, unless the study medication is considered to have contributed to the new AE.

### Pharmacokinetic Assessments/Pharmacodynamic Assessments

Whole blood samples and optional tumor biopsies will be collected at the time points described in Appendix A (Section Error! Reference source not found.). Examples of analysis include relevant immune effector cytokines, target engagement, testing of circulating tumor DNA (ctDNA).

### Disease Progression

Removal of Patients from the Study

Every effort within the bounds of safety and patient choice should be made to have each patient complete the treatment period of the study. Patients who have treatment discontinued due to PD may be treated with any additional therapy deemed appropriate by the investigator.

Patients may be discontinued from the study for any of the following reasons:
Investigator recommends discontinuation and documents the reason(s)
There is a need for any treatment not allowed by the protocol
Patient's decision to withdraw consent or discontinue for any reason
There is an unacceptable AE thought to be related to study medication

Any patient who discontinues during the treatment period should return to complete safety and disease assessments (see Appendix A]).

### Study Completion

The study will be considered complete when all patients have been followed to disease progression; are lost to follow-up, death, or withdrawal due to toxicity; patient's request; or investigator's discretion; and have completed all end-of-study treatment procedures.

### Study Medication

Study medication will be administered in 21-day cycles. Either Talabostat mesylate or pembrolizumab may be administered first. However, on Cycle 1 Day 1, it is recommended that pembrolizumab be administered first and that ≥1 hour should elapse before the administration of Talabostat mesylate so that it will be easier to determine the relatedness of any AEs to study drug.

### Talabostat Mesylate Dosage and Administration

Talabostat mesylate tablets contain valine-proline boronic acid formulated as the methanesulfonate salt. Current dosage strengths include 0.05 mg and 0.2-mg tablets for oral administration.

Talabostat mesylate will be administered orally as a 0.2 mg tablet. Patients will take 3 tablets daily, on days 1 to 14 of each cycle, for a total daily dose of 0.6 mg. Talabostat mesylate will be continued until disease progression or unacceptable toxicity.

On days when pharmacodynamic studies are being performed, Talabostat mesylate should be administered at the study center, and should be administered at (approximately) the same time of day on each treatment day in the cycle. In cycles in which pharmacodynamics are not evaluated, Talabostat mesylate also should be administered at (approximately) the same time of day on each treatment day in the cycle, preferably 0800 hours. If the patient forgets to take study medication the dose will be skipped.

### Dose Adjustments of Talabostat Mesylate Secondary to Toxicity

Talabostat mesylate dose modifications within a treatment cycle are discouraged in Cycle 1 unless required by AE and/or DLT. In Cycle ≥2, dose modifications within a treatment cycle will be at the discretion of the investigator. Doses held because of AEs should not be made up on subsequent days within or following a cycle. A dose that is missed for reasons other than an AE (i.e., the patient forgets to take a dose) may be administered on days subsequent to scheduled doses; any such adjustments should be discussed with the Investigator. Under no circumstances should missed doses be made-up on a day when the patient is already taking a planned dose (i.e., no “doubling-up” to account for missed doses).

Recommendations for Talabostat mesylate dose modifications are:
Grade 2 or higher AEs of edema/peripheral swelling, hypotension, dizziness, and hypovolemia:
Hold Talabostat mesylate until resolution of these AEs to ≤Grade 1 or baseline.
Restart Talabostat mesylate at the full dose after resolution of these AEs to ≤Grade 1 or baseline, including Grade 2 edema.
For any Grade 3 or higher edema or Grade 2 edema that has not improved within 7 days, restart Talabostat mesylate at a dose reduced by 0.2 mg (1 dose level reduction) after resolution of the edema to ≤Grade 1 or baseline. If Grade 2 or greater edema does not recur during the next dosing period (2 weeks on/1 week off), the dose of Talabostat mesylate can be re-escalated at the Investigator's discretion.
For other Grade 2 or higher AEs deemed related to Talabostat mesylate:
Hold Talabostat mesylate until resolution of these AEs to ≤Grade 1 or baseline.
Restart Talabostat mesylate at the full dose or at a dose reduced by 0.2 mg (1 dose level reduction) at the discretion of the Investigator.
A maximum of 2 dose reductions per participant will be permitted for Talabostat mesylate-related AEs, after which the study drug will be permanently discontinued.
Adverse events deemed related to Talabostat mesylate not recovering to ≤Grade 1 or baseline within 6 weeks from onset will require permanent discontinuation of Talabostat mesylate.

If an SAE thought to be related to Talabostat mesylate occurs during the Treatment Period, dosing of Talabostat mesylate should be interrupted in that patient until the SAE resolves. If the Investigator wishes to continue the patient on Talabostat mesylate, the supporter should be contacted to discuss continuing Talabostat mesylate at the same or reduced dose.

If Talabostat mesylate is discontinued due to an AE, all termination from treatment procedures and assessments must be performed.

Monitoring of Patient Compliance with Talabostat Mesylate Study Medication

All Talabostat mesylate dosing containers must be returned to the clinic at each visit. Patients should be queried regarding their compliance with the dosing regimen and medication containers should be reviewed at each visit to determine if any doses of Talabostat mesylate have been missed, and the number of missed doses recorded. Patients must be at least 70% compliant with taking Talabostat mesylate in Cycles 1 and 2 in order to be included in the per-protocol efficacy analyses.

### Talabostat Mesylate Description and Storage

Talabostat mesylate is supplied as 0.05 mg and 0.2-mg tablets in high-density polyethylene bottles with desiccant and child-resistant caps. 30 tablets will be provided in each bottle. Supplies of Talabostat mesylate will be appropriately labeled for clinical trial material. Talabostat mesylate should be stored under refrigerated conditions between 2° C. to 8° C. (36° F. to 46° F.).

### Pembrolizumab Administration, Dose Modifications and Discontinuation

Pembrolizumab will be prepared, stored, and administered according to the current full Prescribing Information. Pembrolizumab will be obtained from commercial supplies and will be administered 200 mg intravenously over 30 minutes through a 0.2 to 5 micron sterile, nonpyrogenic, low-protein binding online or add-on filter. No other medication will be infused through the infusion line. Infusion will be interrupted and slowed for grade 1 or 2 infusion-related reactions and permanently discontinued for grade 3 or 4 infusion-related reactions. Pembrolizumab will be administered until disease progression, unacceptable toxicity, or withdrawal of consent.

AEs associated with pembrolizumab exposure may be immune-mediated. Immune-related AEs may occur any time after pembrolizumab administration and may affect multiple body systems. Early recognition and treatment are important to reduce complications. Most immune-related AEs are reversible and can be managed with discontinuation of pembrolizumab and initiation of steroids. Refer to the current regional pembrolizumab full Prescribing Information for recommended dose modifications for the management of toxicities (including immune-mediated reactions and infusion-related reactions) considered related to pembrolizumab. Patients who require a dose hold of pembrolizumab of ≥42 days will be discontinued from the study.

Pembrolizumab should not be used in conjunction with other immunosuppressive agents other than corticosteroids administered for control of immune reactions considered related to pembrolizumab. Refer to the current regional pembrolizumab full Prescribing Information for further details.

### Data Analysis and Statistical Considerations

This is a phase 2, single center, basket study of oral Talabostat mesylate daily on days 1-14 in combination with intravenous PD1/PDL1 antibody on day 1 of 21-day cycle in subjects with advanced and refractory malignancies. Lead-in cohort will enroll 6 patients. Only the 6 patients treated at the selected dose during safety lead-in will be assigned to Cohort A or B as appropriate. That is, if there is a dose de-escalation during safety lead-in, then the 6 patients treated at the higher dose will not be assigned to the phase II cohorts. Cohorts A and B will enroll 9 to 17 patients. Response assessments with CT and/or MRI will be done every 9 weeks (3 cycles) following RECIST and iRECIST criteria.

The study will follow the BOP2 design with the following operating characteristics.

### Power: 0.80

Type I error: 0.05

### P0: 0.05

### P1: 0.25

Each cohort will enroll 9 patients. If there is no complete (CR) or partial response (PR) in the first 9 patients the enrolment to that cohort will stop. If there ≥1 PR or CR in the first 9 patients the enrollment will continue to enroll total of 17 patients. The treatment will be considered promising for further exploration if ≥3 CRs or PRs are observed in 17 patients. The expected Sample Size will range from 9 (if terminated after safety lead in) to 34 patients. Accounted for ˜20% of patients not being evaluable for efficacy, the actual number of patients to be recruited for the trial will range from 11 to 42.

### Operating Characteristics:

| | | | Average | Average |
|---|---|---|---|---|
| Scenarios | Pr(reject H0 | Pr(reject H0 | sample size | sample size |
| (PA, PB) | for cohort A) | for cohort B) | for cohort A | for cohort B |
| (0.05, 0.05) | 0.046 | 0.046 | 12.0 | 12.0 |
| (0.05, 0.25) | 0.046 | 0.813 | 12.0 | 16.4 |
| (0.25, 0.25) | 0.813 | 0.813 | 16.4 | 16.4 |
| (0.05, 0.15) | 0.046 | 0.455 | 12.0 | 15.1 |
| (0.15, 0.15) | 0.455 | 0.455 | 15.1 | 15.1 |
| (0.15, 0.25) | 0.455 | 0.813 | 15.1 | 16.4 |

Note:
PA is the response rate for cohort A, PB is the response rate for cohort B.

The Investigator is responsible for completing toxicity/efficacy summary reports and submitting them to the IND office Medical Affairs and Safety Group for review. These should be submitted as follows:
Lead-In Stage:

After the first 6 evaluable patients, complete cycle 1 of study treatment. IND Office approval must be obtained prior to advancing to the efficacy stage.
Efficacy Stage:

After the first 9 evaluable patients per cohort, complete 9 weeks of study treatment, and after a total of 17 patients per cohort have completed 9 weeks of study treatment. A copy of the cohort summary should be placed in the Investigator's Regulatory Binder under “sponsor correspondence”.

Toxicity monitoring is also performed in this stage. If the empirical DLT rate >35%, we will suspend accrual for safety and discussions for the next step will be made.

### Safety Analyses

All patients in the safety population will be included in the final summaries and listings of safety data for the lead-in patients.

Frequencies of patients experiencing at least 1 AE will be displayed by body system and preferred term according to Medical Dictionary for Regulatory Activities (MedDRA) terminology. Detailed information collected for each AE will include: a description of the event, duration, whether the AE was serious, nature of the event (single episode versus multiple episode), intensity (i.e., NCI CTCAE version 5 grade), relationship to study drug, action taken, clinical outcome, and whether the AE resulted in surgery or alternate procedures. Intensity (severity) of the AEs will be graded according the NCI CTCAE. The latest version of MedDRA and NCI CTCAE will be used.

Summary tables will be prepared to show the number of patients reporting AEs, the frequency of patient reports, and corresponding percentages. Percentages will be calculated using the number of patients in the safety population as the denominator. Within each table, the AEs will be categorized by MedDRA body system and preferred term. Additional subcategories will be based on event intensity and relationship to study drug. AE data will be presented across all cycles and for each cycle. The denominator for each cycle is those patients available at the start of the cycle who received a dose of Talabostat mesylate for that cycle.

To the extent possible, AE relationship to either or pembrolizumab will be identified.

Individual patient listings will be prepared for all AE data. Talabostat mesylate Vital signs and ECOG performance status will be summarized by visits/cycles, using descriptive statistics applicable to continuous or categorical measures of these additional safety data. Summaries for the Lead-in and Efficacy Stages will be presented.

### Clinical Laboratory Analyses

All clinical laboratory values will be listed individually and tabulated in a manner to identify safety concerns on a per-patient basis. Listing tables will be prepared for each laboratory measure and will be structured to permit review of the patient data as they progress on treatment. The tables will list the cycle of treatment, Talabostat mesylate dose for Lead-in data, and the associated NCI CTCAE grade. Descriptive summary statistics will be generated per laboratory parameter.

Summary tables will be prepared to examine the distribution of these toxicities per cycle.

Graphic displays and shift tables may be provided to illustrate results over time on study. Assessment of cumulative toxicities may be made.

### Investigator Requirements

### Protocol Adherence

Each investigator must adhere to the protocol as detailed in this document and agree that any intended departures from the protocol must be approved by the Principal Investigator or her/his designee prior to seeking approval from the IRB. Each investigator will be responsible for enrolling only those patients who have met protocol eligibility criteria.

### Study Monitoring Requirements

Site visits will be conducted by the sponsor or sponsor's representative to inspect study data, patients' medical records, and other documents in accordance with current Food and Drug Administration (FDA) Good Clinical Practices (GCP), the International Council for Harmonisation (ICH) guidelines, and the respective local and national government regulations and guidelines. The investigator will permit the sponsor and/or authorized representatives of the sponsor, the FDA, and the respective national or local health authorities to inspect his or her facility and records relevant to this study.

### Drug Accountability

Inventory control of all Talabostat mesylate must be maintained throughout the duration of the study. Any discrepancies that are noted between drug-dispensing records and drug inventory must be reported. Medication-dispensing records are provided to the investigative site. All study medication used during the study must be accounted for on the appropriate form. All unused study medication must be returned by the patient to the site for completion of their drug accountability record. All unused study medication will be disposed of in biohazard containers in accordance with the policies of the institution by site personnel.

### Retention of Records

Records and documents pertaining to the conduct of this study, including screening logs, source documents, consent forms, laboratory test results, medication inventory records and other documents must be retained according to the local standard operating procedures and institutional and/or IRB policies.

### Study Discontinuation

### 1 Ethical Considerations

This study will be conducted in accordance with current FDA regulations, GCP, the ICH guidelines, the ethical principles that have their origins in the Declaration of Helsinki, and local ethical and legal requirements.

1. A treatment regimen for treating prostate cancer in a subject in need thereof, the regimen comprising administering to said subject, as separate pharmaceutical formulations, an effective amount of Talabostat or a pharmaceutically acceptable salt thereof and an effective amount of Pembrolizumab, wherein Talabostat or a pharmaceutically acceptable salt thereof and Pembrolizumab are administered in one or more treatment cycles and each treatment cycle is of about 21 days duration.

2. A method of treating prostate cancer in a subject in need thereof, the method comprising administering to the subject, as separate pharmaceutical formulations, an effective amount of Talabostat or a pharmaceutically acceptable salt thereof and an effective amount of Pembrolizumab, wherein Talabostat or a pharmaceutically acceptable salt thereof and Pembrolizumab are administered to the subject in 1, 2 or 3 treatment cycles and each treatment cycle is of about 21 days duration.

3. (canceled)

4. (canceled)

5. The treatment regimen according to claim 1, wherein the prostate cancer is small cell neuroendocrine prostate cancer (SCNC).

6. The treatment regimen according to claim 1, wherein after cessation of treatment the subject maintains a sustained response to progression of prostate cancer.

7. The treatment regimen according to claim 1, wherein for each treatment cycle Talabostat or a pharmaceutically acceptable salt thereof is administered on each of days 1 to 14 and Pembrolizumab is administered on day 1.

8. The method according to claim 2, wherein for each treatment cycle Talabostat or a pharmaceutically acceptable salt thereof is administered on each of days 1 to 14 and Pembrolizumab is administered on day 1.

9. The treatment regimen according to claim 1, wherein Talabostat or a pharmaceutically acceptable salt thereof is administered orally in one or more tablet.

10. The treatment regimen according to claim 1, wherein Pembrolizumab is administered by intravenous injection.

11. The treatment regimen according to claim 1, wherein Talabostat or a pharmaceutically acceptable salt thereof is administered at a total daily dose of from about 0.001 mg/kg to about 0.1 mg/kg.

12. The treatment regimen according to claim 1, wherein Pembrolizumab is administered at a total dose of from about 1 mg/kg to about 10 mg/kg per day.

13. The treatment regimen according to claim 1, wherein Talabostat or a pharmaceutically acceptable salt thereof is administered at a total daily dose of from about 0.4 mg to about 0.6 mg.

14. The treatment regimen according to claim 1, wherein Pembrolizumab is administered at a total dose of from about 100 mg to about 500 mg per day.

15. The treatment regimen according to claim 1, wherein Pembrolizumab is administered at a total dose of about 200 mg per day.

16. The treatment regimen according to claim 1, wherein the total daily dose of Talabostat in cycle 1 is lower than the total daily dose of Talabostat or a pharmaceutically acceptable salt thereof in one or more subsequent cycles.

17. The treatment regimen according to claim 1, wherein Talabostat or a pharmaceutically acceptable salt thereof and Pembrolizumab are administered to the subject in one or more treatment cycles, where each treatment cycle is of about 21 days duration, and for each treatment cycle Talabostat or a pharmaceutically acceptable salt thereof is administered on each of days 1 to 14 and Pembrolizumab is administered on day 1.

18. The treatment regimen or method according to claim 17, wherein Talabostat or a pharmaceutically acceptable salt thereof is administered at a total daily dose of from about 0.4 mg to about 0.6 mg.

19. The treatment regimen or method according to claim 18, wherein the total daily dose of Talabostat or a pharmaceutically acceptable salt thereof in cycle 1 is lower than the total daily dose of Talabostat in one or more subsequent cycles.

20. The treatment regimen according to claim 1, wherein Talabostat or a pharmaceutically acceptable salt thereof is Talabostat mesylate.

21. A treatment regimen for treating prostate cancer in a subject in need thereof, the regimen comprising administering to the subject Talabostat mesylate and Pembrolizumab in one or more treatment cycles, where each treatment cycle is of about 21 days duration, and for each treatment cycle Talabostat is administered on each of days 1 to 14 and Pembrolizumab is administered on day 1, wherein Talabostat mesylate is administered as one or more tablets to provide a total daily dose of Talabostat of from about 0.4 mg to about 0.6 mg and Pembrolizumab is administered as a single intravenous injection to provide a dose of from about 100 mg to about 500 mg per day.
